(19) United States
(12) Patent Application Publication
(10) Pub. No.: US 2024/0285554 A1
(43) Pub. Date: Aug. 29, 2024

(54) POLYVINYL ALCOHOL COMPOSITIONS AND METHODS OF MAKING AND USING THEM

(71) Applicant: Emanate Biomedical, Inc., Larchmont, NY (US)

(72) Inventors: PETER JOHN ZEGARELLI, Sleepy Hollow, NY (US); Dorna Hakimimehr, Redwood City, CA (US); Jarret Scott Fass, Larchmont, NY (US); Tom Huljev, San Carlos, CA (US)

(73) Assignee: Emanate Biomedical, Inc., Larchmont, NY (US)

(21) Appl. No.: 18/114,559

(22) Filed: Feb. 27, 2023

(51) Int. Cl. A61K 31/155; A61C 19/06; A61K 47/10; A61K 47/34; C08K 5/053; C08K 5/29

(52) U.S. Cl. CPC A61K 31/155; A61C 19/063; A61K 47/10; A61K 47/34; C08K 5/053; C08K 5/29

(57) ABSTRACT

A composition for treating an oral cavity in a patient in need is provided. The composition comprising a medicament, a polyvinyl alcohol (PVA), and optionally a humectant, the medicament in an amount of about 0.001% w/w, v/v or w/v to about 5% w/w, v/v or w/v, the polyvinyl alcohol in an amount of about 5% w/w, v/v or w/v to about 99% w/w, v/v or w/v, the humectant in an amount of about 0.5% w/w, v/v or w/v to about 15% w/w, v/v or w/v based on a total w/w, v/v or w/v of the composition. A method of making a composition for treating an oral cavity in a patient in need is also provided.

### BACKGROUND

Oral dosage forms (e.g., tablet, capsules, suspensions, etc.) account for approximately eighty percent of all medicament dosage forms on the market. Oral dosage forms are non-invasive, easily administered, have high patient compliance and are often used to treat acute and chronic systemic conditions (e.g., infection, diabetes, high cholesterol, heart disease, etc.).

Orally administered medicaments can be rapidly transported to the stomach and small intestine for absorption across the gastrointestinal (GI) mucosal membranes into the blood. The efficiency of absorption of a medicament following oral administration can be low because of metabolism within the GI tract and first-pass metabolism within the liver resulting in relatively lengthy onset times or erratic absorption characteristics that may not be well suited to control acute systemic disorders. The majority of oral dosage forms on the market are designed for GI delivery to treat acute and chronic systemic conditions.

One area of recent interest is medicament delivery using the oral tissue (e.g., gingival tissue) of the oral cavity. Due to the avoidance of the first-pass metabolism and avoidance of inefficient medicament absorption through the gut, medicament delivery using oral tissue (e.g., gingival tissue) may improve the medicament's bioavailability, when a systemic effect is desired. It therefore can be advantageous for a medicament to be delivered using the gingiva of the oral cavity, when rapid onset, consistent Tₘₐₓ and Cₘₐₓ are advantageous.

There are also non-systemic or localized conditions of the oral cavity that can also benefit from delivery of medicament to the oral cavity. These conditions include, but are not limited to, oral wounds, gum disease, gingivitis and periodontitis.

Wounds in the oral cavity undergo complex sequences of biological processes to restore homeostasis and healing. While general similarities exist, there are marked differences in the genomics and kinetics of wound healing between the oral cavity and cutaneous epithelium. The lack of successful therapy for oral mucosal wounds has influenced clinicians to explore alternative treatments for wound healing.

Gingivitis and periodontitis are two common gum diseases that can benefit from wound healing treatments. Dental plaque is a precursor of calculus. Plaque is recognized as a precursor of such oral diseases as caries and gum disease. Gum disease (gingivitis and periodontitis) is an infectious disease characterized by inflammation of the gums in response to a build-up of pathological bacteria around the teeth and gums. The bacteria associated with plaque can secrete enzymes and endotoxins, which can irritate the gums and cause gingivitis. As the gums become increasingly irritated by this process they tend to bleed, lose their toughness and resiliency, and separate from the teeth, leaving periodontal pockets in which debris, secretions, more bacteria and toxins further accumulate. In periodontitis, the gums recede as the periodontal ligament is damaged. This leads to the formation of periodontal pockets. These pockets host a higher percentage of pathologic anaerobic bacteria that cause more inflammation than in healthy gums. The “red complex” bacteria found in periodontal pockets are inflammophillic—that is, they feed off of the inflammatory tissue breakdown products. This dysbiosis, or imbalance in relative abundance or influence of pathologic bacteria, becomes self-perpetuating in a positive feedback loop. Eventually, the degradation of the supporting structures of the teeth, including alveolar bone, leads to bone loss, which when severe can lead to the eventual loss of the tooth.

In treating gum disease, consideration should be given to treat the infection. Current treatment of periodontal disease consists of scaling and root planning, which is an oftentimes painful, invasive procedure that mechanically removes the bacteria from the pockets and smooths the surface of the root of the tooth to help prevent bacteria from building up. Since patients cannot access deep pockets easily by flossing or brushing, they are required to return to the dentist/periodontist for routine periodontal maintenance therapy, on average every 3 to 6 months. Periodontal disease, though widely recognized as having a bacterial etiology, is often still being treated by mechanical procedures alone (see J. Max Goodson, Antimicrobial strategies for treating periodontal diseases, Periodontology, Vol. 5, 1994).

Chlorhexidine rinse is one type of oral medicament used in the treatment of gingivitis and periodontitis. Chlorhexidine rinse is often prescribed as an adjunct to scaling and root planning. It is a broad-spectrum antimicrobial that can be effective against pathologic red complex bacteria. Often times, for chlorhexidine to be effective, it can have a minimum inhibitory concentration (MIC) when the antimicrobial is in direct contact with the bacteria. This is the concentration at which the bacteria's growth is inhibited. Minimum bactericidal concentration (MBC) is the concentration at which the bacteria are irreversibly killed, after a qualified amount of time. Chlorhexidine's MIC against oral pathogenic bacteria is 0.0125% w/v, and its MBC is 0.5% w/v at 10 minutes. By inhibiting the growth of bacteria, the host is able to recover and heal, breaking the pathogenic cycle. Because of chlorhexidine's cationic binding properties, a 0.12% w/v oral rinse maintains MIC for 4 hours after a 30 second rinse. Unfortunately, chlorhexidine rinse does not reach the desired MIC concentration subgingivally and causes other local adverse effects such as stained teeth, unpleasant taste and black tongue. There is little proven clinical benefit of the chlorhexidine rinse for periodontitis.

Oral chlorhexidine rinse treatments suffer from additional drawbacks. For example, it is well known that chlorhexidine rinses have an extremely bitter taste. This objectionable taste causes major compliance problems, particularly with pediatric patients. In order to combat the bitter taste, certain flavoring agents and sweetening agents have been added to chlorhexidine rinses. Additionally, chlorhexidine is a strong basic material that reacts with a wide variety of compounds and chemical structures that are often used in the commercial production of chlorhexidine products. For example, the addition of many flavoring agents, which are often aldehydes in structure, or sweetening agents can reduce or eliminate the antimicrobial activity of chlorhexidine via chemical reactions that include salt formation and precipitation.

Recently, there has been some interest in using hydrogels in medicament delivery. Hydrogels are three-dimensional water-swollen polymers that are biocompatible, and possess mechanical strength. Hydrogels are highly porous and can accommodate various loads of medicaments. Typically, hydrogels are used in skin wound dressings, tissue engineering, and ocular compositions.

There is a need for new and improved hydrogel compositions suitable for use in the oral cavity that have the medicament (e.g., chlorhexidine) loaded within them to help heal oral wounds as well as treat various local and systemic conditions. These hydrogel compositions containing medicament can be loaded in an oral appliance, maintain a moist environment, support oral wound healing (e.g., periodontal pockets, canker sores, gum abrasions, etc.), or be used to treat systemic conditions. It would also be beneficial to provide hydrogel compositions having suitable viscosities to allow the hydrogel compositions to be attached to or be held within the oral appliance with little or no leakage from the oral appliance before it is worn by the patient.

### SUMMARY

New and improved hydrogel compositions are provided that are suitable for use in the oral cavity that have the medicament (e.g., chlorhexidine) loaded within them to help heal oral wounds as well as treat various local and systemic conditions. In some embodiments, the hydrogel compositions containing medicament can be loaded in an oral appliance, maintain a moist environment, support oral wound healing (e.g., periodontal pockets, canker sores, gum abrasions, etc.), or be used to treat systemic conditions (e.g., infection, diabetes, high cholesterol, heart disease, etc.). In some embodiments, the hydrogel compositions are processed to acquire suitable viscosities to allow the hydrogel compositions to be loaded to or be held within the oral appliance with little or no leakage before it is worn by the patient.

In some embodiments, there is a composition for treating an oral cavity in a patient in need thereof, the composition comprising a medicament, polyvinyl alcohol (PVA), water, and optionally a humectant. The medicament can be in an amount of about 0.001% w/w, v/v or w/v to about 5% w/w, v/v or w/v, the polyvinyl alcohol in an amount of about 5% w/w, v/v or w/v to about 99% w/w, v/v or w/v or about 50% w/w, v/v, or w/v, the humectant in an amount of about 0.5% w/w, v/v or w/v to about 15% w/w, v/v or w/v based on a total w/w, v/v or w/v of the composition.

In some embodiments, there is a composition for treating an oral cavity in a patient in need thereof, the composition comprising a polyvinyl alcohol (PVA), and a humectant, the polyvinyl alcohol in an amount of about 5% w/w, v/v or w/v to about 99% w/w, v/v or w/v or about 50% w/w, v/v, or w/v, the humectant in an amount of about 0.5% w/w, v/v or w/v to about 15% w/w, v/v or w/v based on a total w/w, v/v or w/v of the composition.

In some embodiments, there is a composition for treating an oral cavity in a patient in need thereof, the composition comprising a medicament, glycerol, polyvinyl alcohol and water in the composition or water in the polyvinyl alcohol, the medicament being in an amount of about 0.001% to about 20% w/w, v/v or w/v, the glycerol being in an amount of from about 5% to about 9% w/w, v/v or w/v, polyvinyl alcohol being in an amount from about 12% to about 19% w/w, v/v or w/v, and water being in the composition or in the polyvinyl alcohol in an amount from about 61% to about 82% w/w, v/v or w/v based on a total w/w, v/v or w/v of the composition.

In some embodiments, there is a method of making a composition for treating an oral cavity in a patient in need thereof, the method comprising dissolving PVA in water to form a mixture; adding in any order about 0.001% to about 20% w/w, v/v or w/v of a medicament, and about 5% to about 9% w/w, v/v or w/v glycerol based on a total w/w, v/v or w/v of the mixture to make the composition, and optionally crosslinking the PVA to form a hydrogel.

In some embodiments, there is a composition for treating the oral cavity in a patient in need thereof, the composition comprising a preservative, a polyvinyl alcohol (PVA), and optionally a humectant, the preservative in an amount of about 0.001% w/w, v/v or w/v to about 5% w/w, v/v or w/v, the polyvinyl alcohol in an amount of about 5% w/w, v/v or w/v to about 99% w/w, v/v or w/v, the humectant in an amount of about 0.5% w/w, v/v or w/v to about 15% w/w, v/v or w/v based on a total w/w, v/v or w/v of the composition.

In some embodiments, there is a composition for treating an oral cavity in a patient in need thereof, the composition comprising a preservative, glycerol, polyvinyl alcohol and water in the composition or water in the polyvinyl alcohol, the preservative being in an amount of about 0.001% to about 20% w/w, v/v or w/v, the glycerol being in an amount of from about 5% to about 9% w/w, v/v or w/v, polyvinyl alcohol being in an amount from about 12% to about 19% w/w, v/v or w/v, and water being in the composition or in the polyvinyl alcohol in an amount from about 61% to about 82% w/w, v/v or w/v based on a total w/w, v/v or w/v of the composition.

In some embodiments, there is a method of making a composition for treating an oral cavity in a patient in need thereof, the method comprising mixing in any order about 12% to about 19% w/w, v/v or w/v of polyvinyl alcohol with about 0.001% to about 20% w/w, v/v or w/v of a preservative, and about 5% to about 9% w/w, v/v or w/v glycerol based on a total w/w, v/v or w/v of the composition to make the composition.

In one embodiment, after the addition of the medicament and optionally glycerol to the PVA and water mixture, the PVA can be chemically crosslinked and/or physically crosslinked to form the hydrogel. Chemical crosslinking can be done by adding a crosslinking agent to the PVA and water mixture, the PVA and water mixture can have medicament and optionally glycerol disposed within it. Chemical crosslinking agents include, but are not limited to, aldehydes, such as for example glutaraldehyde (GA), sodium trimetaphosphate (STMP), or a combination thereof.

In another embodiment, the PVA in the PVA and water mixture having medicament and optionally glycerol disposed in it can be physically crosslinked. One example of physical crosslinking includes, but is not limited to, using freeze-thaw (FT) cycles. The PVA can be subjected to multiple FT cycles in order to produce the hydrogel having the desired properties.

In one embodiment of the present application, the hydrogel is embedded on the oral appliance by loading the oral appliance with the hydrogel and then the hydrogel is crosslinked. In another embodiment, the hydrogel is first crosslinked before being loaded onto the dental appliance.

In one embodiment the viscosity of the PVA hydrogel is lowered by overnight freezing and thawing before filling the dental appliance with the hydrogel. The hydrogel is then further crosslinked using physical or chemical means.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE FIGURES

In part, other aspects, features, benefits and advantages of the embodiments will be apparent with regard to the following description, appended claims and accompanying drawings.

FIG. 1 illustrates an enlarged side view of an embodiment of the oral appliance configured to contour at least a portion of the teeth and/or soft tissues in the oral cavity of a patient, the oral appliance is shown without teeth and/or soft tissues inserted in the oral appliance.

FIG. 2 illustrates an enlarged side view of an embodiment of the oral appliance, where the medicament (e.g., chlorhexidine) is shown disposed in a hydrogel (e.g., poly vinyl alcohol), which is disposed at discrete regions of the oral appliance adjacent to an infected tissue or oral wound, which is shown at the gingival sulcus region. This view has the teeth and gums loaded in the interior of the oral appliance. It will be understood by those of ordinary skill in the art that the hydrogel composition can be uniformly loaded with medicament and this hydrogel can be disposed uniformly throughout the oral appliance or at discrete region(s) of the oral appliance depending on where the treatment area is located.

In some embodiments, the hydrogel can have medicament disposed at discrete regions of the hydrogel, where the hydrogel can then be disposed at discrete regions of the oral appliance. For example, the hydrogel can have a discrete region or discrete regions containing medicament, which then can be disposed at discrete regions of the oral appliance. In this way, specifically targeted medicament delivery to discrete regions of the oral cavity adjacent to the oral appliance can be controlled to limit or reduce unwanted off-target effects and provide additional comfort to the wearer even at regions of the oral cavity that do not require treatment as the hydrogel by itself, with or without medicament, provides a nice comfortable watery and absorbent cushion for the wearer of the oral appliance to experience.

FIG. 3A illustrates an enlarged view of the composition that is to be placed inside an oral appliance. Here the composition comprises a hydrogel that contains the medicament (e.g., chlorhexidine) uniformly disposed throughout it. Shown here is the hydrogel that is crosslinked and a semisolid structure.

FIG. 3B illustrates an enlarged interior view of the oral appliance having the composition containing the hydrogel disposed in a channel of the oral appliance that can be a discrete region that contours the sulcus (gumline) and the medicament (e.g., chlorhexidine) targets wounds of the gum. Here the oral appliance is shown as a custom fit oral appliance that is placed adjacent to the teeth and/or soft tissue areas of the oral cavity.

FIG. 4 illustrates an enlarged cross-sectional view of the anatomy of the gums and a tooth including free gingiva, attached gingiva, lining mucosa, the alveolar bone, the periodontal pocket or crevice, the cementoenamel junction (CEJ), periodontal ligament, cementum, the enamel, dentin, pulp and the junctional epithelium. In some embodiments, the periodontal pocket is the wound that is targeted for medicament delivery and the oral appliance and hydrogel containing medicament are place adjacent to this periodontal pocket. The design of the medicament (e.g., chlorhexidine) composition of the oral appliance is to target the periodontal pocket or crevice of the sulcus and extrude the medicament (e.g., chlorhexidine) into the entrance of the periodontal pocket.

FIG. 5 illustrates an enlarged cross-sectional view of a portion of the oral appliance. In the embodiment shown, a medicament (e.g., chlorhexidine) is disposed in a porous material that is a hydrogel at a discrete region of the oral appliance. The hydrogel is shown in an uncompressed state and when worn with slight pressure, the hydrogel will be compressed against, among other things, the gingival crevice or periodontal pocket causing a seal of the entrance of the gingival crevice or periodontal pocket, which prevents oral fluids (e.g., saliva, exudate, or other captured foreign fluids from insertion of the device, etc.) from entering the crevice or pocket, which allows release of the chlorhexidine in the gingival crevice or periodontal pocket and allows the hydrogel to absorb or wick fluid from the crevice or pocket.

FIG. 6 illustrates an enlarged cross-sectional view of a portion of the oral appliance that is placed adjacent to the teeth and gums. In the embodiment shown, a medicament (e.g., chlorhexidine) is disposed in a porous material that is a hydrogel at a discrete region of the oral appliance. The hydrogel is shown in a compressed state, where the device is worn and the hydrogel is compressed against, among other things, the gingival crevice or periodontal pocket causing a seal or encapsulation of the entrance of the gingival crevice or periodontal pocket, which prevents oral fluids (e.g., saliva, exudate, or other captured fluids upon insertion of the device, etc.) from entering the crevice or pocket. The hydrogel allows release of the medicament (e.g., chlorhexidine) into the gingival crevice or periodontal pocket to treat the inflamed tissue shown by the down arrows. The hydrogel also absorbs or wicks oral fluids from the crevice or pocket, which aides healing, shown by the up arrows.

FIG. 7 illustrates an embodiment of a flow chart of the preparation of the hydrogel composition (e.g., PVA containing chlorhexidine gluconate) configured for the delivery via a device or an oral appliance.

FIG. 8 illustrates another embodiment of a flow chart of the preparation of the hydrogel composition (e.g., PVA containing chlorhexidine) configured for the delivery via a device or an oral appliance.

It is to be understood that the figures are not drawn to scale. Further, the relationship between objects in a figure may not be to scale and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a humectant” includes one, two, three or more humectants.

A “carrier” is any substrate used in the process of delivering the medicament (e.g., chlorhexidine). The carrier serves to improve the selectivity, effectiveness, and/or safety of chlorhexidine administration. Carriers can be used to control the release of a medicament (e.g., chlorhexidine). This can be accomplished either by slow release of the medicament (e.g., chlorhexidine) over a long period of time (typically diffusion) or by triggered release at the medicament's target by some stimulus, such as, for example, changes in pH, application of heat, activation by light, physical stimulus such as the hydrogel being squeezed against the oral tissue and released, etc. Suitable carriers include one or more polymers, such as for example, hydrogels.

The term “porous” as used herein, refers to a carrier which is permeable such that fluids are movable therethrough by way of pores or other passages. An example of a porous material is a hydrogel material, a cellulosic material, concrete, ceramics, foams, sponges and derivatives thereof. The porous material may be the result of using a low or high molecular weight polymer. In some embodiments, the polymer may be porous as it is dispensed at a low viscosity on the oral appliance and/or substrate, or is dispensed in a geometric pattern, either as a specific structure or a randomized structure.

The term “non-porous” as used herein, refers to a material which is impermeable such that fluids cannot move through the material. Typically, the oral appliance is made from non-porous material, which can be a high density polymer.

The term “hydrogel” or “hydrogels” refer to a broad class of polymeric materials, that may be natural or synthetic, which have an affinity for an aqueous medium, and are able to absorb aqueous medium, but which do not normally dissolve in the aqueous medium. Typically, the oral appliance is loaded with hydrogel. The hydrogel may or may not contain medicament.

The term “dental plaque” is a general term for the diverse microbial community (predominantly bacteria) found on the tooth surface, embedded in a matrix of polymers of bacterial and salivary origin.

The term “oral diseases” refers to diseases and disorders affecting the oral cavity or associated medical conditions. Oral diseases include, but are not limited to, inflammation, infection, dental caries, periodontal diseases (e.g., gingivitis, adult periodontitis, early-onset periodontitis, chronic periodontitis and/or aggressive periodontitis) or the like. Inflammatory diseases can also include benign and malignant tumors such as Lichen Planus and squamous cell carcinoma, respectively, as well as various yeast and fungal infections and conditions like Xerostomia.

The term “gingiva” or “gum” refers to a dense fibrous tissue and overlying mucous membrane enveloping alveolar processes of upper and lower jaws and surrounding the necks of teeth.

The term “gingivitis” refers to inflammation of gingival tissue without loss of connective tissue.

The term “inflamed tissue” refers to bone, teeth, or oral tissue (e.g., gingival tissue) which is red, swollen and can be painful. It will also more broadly include dental caries and/or hypersensitive areas of the teeth. Inflammation can be caused by trauma to the oral cavity, infection or other causes.

The term “periodontal disease” refers to an inflammatory process of the gingival tissues and/or periodontal membrane of the teeth, resulting in a deep gingival sulcus, possibly producing periodontal pockets and loss of alveolar bone.

The term “periodontitis” refers to inflammation and loss of connective tissue of the supporting or surrounding structure of teeth with loss of attachment.

The terms, “treating” or “treatment” includes “preventing” or “prevention” of disease. In addition, “treating” or “treatment” does not require complete alleviation of signs or symptoms, does not require a cure, and specifically includes protocols that have only a marginal effect on the patient.

The term “localized” delivery includes delivery where medicament (e.g., chlorhexidine) contacts the tooth and/or soft tissue areas, for example, the gingival margins of the teeth or a region inside of the mouth such as the palate, or in close proximity thereto.

The term “targeted delivery” includes delivery of medicament (e.g., chlorhexidine) at the target site as needed for treatment of the disease or condition including wound healing. In some embodiments, the oral appliance can be used to deliver medicament (e.g., chlorhexidine) to the soft tissue of the inside of the mouth, including but not limited to any soft tissue adjacent or between the teeth, including but not limited to the papilla, tissue of the upper and lower dental arches, marginal gingiva, gingival sulcus, inter-dental gingiva, gingival gum structure on lingual and buccal surfaces up to and including the muco-gingival junction and/or the palate and/or the floor of the mouth. In various embodiments, the soft tissue area includes the muco-buccal folds, hard and soft palates, the tongue, lining mucosa, and/or attached oral tissue (e.g., gingival tissue).

The term “custom fit” as used herein, refers to an oral appliance that is specifically made via molding and/or 3D printing or other means, to correspond to at least a portion of a tooth, a selected number of teeth, all of the teeth and/or soft tissues found in the mouth of a specific individual patient. A custom fit oral appliance is not a generic device which is then heated or otherwise manipulated by a consumer, inserted into their mouth by themselves and then molded by that consumer to fit their own mouth. The patient image is the result of an action upon that particular individual by another person whereas the consumer is acting upon himself/herself by manually manipulating the generic material. In some embodiments, custom fit includes situations where the patient images himself or herself with a scanning device including those available in smartphone (e.g., I-phone, Android, Galaxy or the like) and then the appliance is made as a separate act.

The term “preservative” includes any chemical that, when added to the composition to reduce microbial loads, suppress their proliferation, or prevent new microbial contamination. The concentration of preservatives in the final dosage form cannot exceed the level that is toxic to humans. In some embodiments, the medicament (e.g., chlorhexidine) used in the current composition can function as a preservative.

The term “antimicrobial” includes an agent that kills microorganisms or stops their growth. In some embodiments, the medicament (e.g., chlorhexidine) used in the current composition can function as an antimicrobial.

The term “wound healing” includes assisting or accelerating to heal a wound surface. In some embodiments, the medicament (e.g., chlorhexidine) used in the current composition can function to enhance wound healing.

The term “humectant” refers to a hygroscopic substance used to keep things moist and it is the opposite of a desiccant. A humectant is often a molecule with several hydrophilic groups, most often hydroxyl groups; however, amines and carboxyl groups, sometimes esterified, can be encountered as well (its affinity to form hydrogen bonds with molecules of water, is the characteristic of a humectant). A humectant attracts and retains moisture in the hydrogel composition to prevent the hydrogel from drying during storage. An example of a humectant that can be used in the medicament composition of the current application includes glycerol.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

The headings below are not meant to limit the disclosure in any way; embodiments under anyone heading may be used in conjunction with embodiments under any other heading.

### Compositions

New and improved hydrogel compositions are provided that are suitable for use in the oral cavity that have the medicament (e.g., chlorhexidine) loaded within them to help heal oral wounds as well as treat various local and systemic conditions. In some embodiments, these hydrogel compositions containing medicament can be loaded in an oral appliance, that hydrate affected oral tissue and can treat locally the oral wound (e.g., periodontal pockets, canker sores, gum abrasions, etc.) or be used to treat systemic conditions (e.g., infection, diabetes, high cholesterol, heart disease, etc.). In some embodiments, the hydrogel compositions have suitable viscosities to allow the hydrogel compositions to be attached to or be held within the oral appliance with little or no leakage from the oral appliance before it is worn by the patient.

In various embodiments, the hydrogel composition is used in conjunction with the oral appliance to manage pain in all types of oral wounds, mouth sores, injuries and ulcers of the oral mucosa. It forms a protective barrier between the wound and further irritation and contamination. It provides a moist wound environment required for optimal wound healing. Examples of oral lesions include canker sores (aphthous ulcers), tooth extraction sites, oral mucositis, oral stomatitis, traumatic ulcers such is caused by braces and dentures, and irritation and pain following deep tooth scaling. In some embodiments, the oral appliance is a single use, nonsterile, patient specific device, provided by a dental professional and worn by the patient following occurrence of a dental wound. In some embodiments, the oral appliance consists of two patient specific dental trays, one for the lower jaw and one for the upper jaw. Each of the trays are designed with an integral channel along the oral wound to be treated by the composition. The channel is pre-filled with a hydrogel. Once worn by the patient, the oral appliance creates a tight fit, gently pressing the hydrogel along the oral wound, thus reducing pain and assisting in wound healing.

The oral appliance contains a hydrogel, which can be located at discrete regions or in a channel on the interior surface, exterior surface or both surfaces of the oral appliance. In many embodiments, the oral composition consists of, consist essentially of, or comprises optionally a medicament, a hydrogel, and optionally a humectant.

In some embodiments, there is a composition for treating an oral cavity in a patient in need thereof, the composition comprising a medicament, a polyvinyl alcohol (PVA), and optionally a humectant, the medicament in an amount of about 0.001% w/w, v/v or w/v to about 5% w/w, v/v or w/v, the polyvinyl alcohol in an amount of about 5% w/w, v/v or w/v to about 50% w/w, v/v or w/v or about 99% w/w, v/v, or w/v, the humectant in an amount of about 0.5% w/w, v/v or w/v to about 15% w/w, v/v or w/v based on a total w/w, v/v or w/v of the composition.

In some embodiments, there is a composition for treating an oral cavity in a patient in need thereof, the composition comprising a polyvinyl alcohol (PVA), and a humectant, the polyvinyl alcohol in an amount of about 5% w/w, v/v or w/v to about 99% w/w, v/v or w/v or about 50% w/w, v/v, or w/v, the humectant in an amount of about 0.5% w/w, v/v or w/v to about 15% w/w, v/v or w/v based on a total w/w, v/v or w/v of the composition.

### Medicaments

The hydrogel composition can comprise a medicament. Examples of medicaments include, but are not limited to, anti-inflammatory agents, anti-infective agents (e.g., antiviral, antibacterial, antifungal agents, etc.), nitric oxide, silver nitrate, chlorhexidine, ACE inhibitors, thyroid agents, diuretics, calcium channel blockers, benzodiazepine, proton pump inhibitors, antiemetics, antipsychotics, PDE5 inhibitors, anticoagulants, z-drugs/hypnotics, anticonvulsants, tissue and bone growth factors, pain management medication (e.g., analgesics, anesthetics, etc.), steroids, leukotriene inhibitors, anti-gout agents, bisphosphonates, antimalarial agents, antihyperlipidemics, calcimimetics, anti-covid agents, antimuscarinics, anticancer agents, birth control agents, NSAIDs, immunomodulators, thiazolidinediones, immunosuppressants, norepinephrine reuptake inhibitors, dopamine antagonists, 5-alpha reductase inhibitors, SNRI agents, antiretroviral, fibrates, monoclonal antibodies, hepatitis C agents, NMDA antagonists, laxatives, stool softeners, antitussives, antidiabetic agents, bronchodilators, DMARDs, acetylcholinesterase inhibitors, antihypertensive agents, alpha-1 blockers, estrogen modulators, antispasmodics, anti-migraine agents, muscle relaxants, nitrates, CNS stimulants, insulin, antihistamines, antineoplastic agents, statins (e.g., atorvastatin, rosuvastatin, etc.), anti-triglycerides, tooth whitening agents, breath fresheners, anticalculus agents, antineoplastic agents, oral dermatologics, selective H-2 antagonists, anticaries agents, beta-blockers, antiparkinson drugs, anti-anxiety agents, antidepressants, muscle relaxants, antibiotics, nutrients, vitamins, minerals, herbal products, opioids, or mixtures thereof. The medicament also includes the preservatives, the antimicrobials and the wound healing agents.

In some embodiments, the oral appliance can have an opioid, or other addictive or abused drug (e.g., medicament) disposed in discrete porous portions or throughout the entire oral appliance. The opioid, or other addictive or abused drug can include, but is not limited to, 1-phenylcyclohexylamine, 1-piperidinocyclohexanecarbonitrile, alfentanil, alphacetylmethadol, alphaprodine, alprazolam, amobarbital, amphetamine, anileridine, apomorphine, aprobarbital, barbital, barbituric acid derivative, bemidone, benzoylecgonine, benzphetamine, betacetylmethadol, betaprodine, bezitramide, bromazepam, buprenorphine, butabarbital, butalbital, butorphanol, camazepam, cathine, chloral, chlordiazepoxide, clobazam, clonazepam, clorazepate, clotiazepam, cloxazolam, cocaine, codeine, chlorphentermine, delorazepam, dexfenfluramine, dextromoramide, dextropropoxyphen, dezocine, diazepam, diethylpropion, difenoxin, dihydrocodeine, dihydromorphine, dioxaphentyl butyrate, dipanone, diphenoxylate, diprenorphine, ecgonine, enadoline, eptazocine, estazolam, ethoheptazine, ethyl loflazepate, ethylmorphine, etorphine, femproponex, fencamfamin, fenfluramine, fentanyl, fludiazepam, flunitrazepam, flurazepam, glutethimide, halazepam, haloxazolam, hexalgon, hydrocodone, hydromorphone, isomethadone, hydrocodone, ketamine, ketazolam, ketobemidone, levanone, levoalphacetylmethadol, levomethadone, levomethadyl acetate, levomethorphan, levorphanol, lofentanil, loperamide, loprazolam, lorazepam, lormetazepam, lysergic acid, lysergic acid amide, mazindol, medazepam, mefenorex, meperidine, meptazinol, metazocine, methadone, methamphetamine, methohexital, methotrimeprazine, methyldihydromorphinone, methylphenidate, methylphenobarbital, metopon, morphine, nabilone, nalbuphine, nalbupine, nalorphine, narceine, nefopam, nicomorphine, nimetazepam, nitrazepam, nordiazepam, normethadone, normorphine, oxazepam, oxazolam, oxycodone, oxymorphone, pentazocine, pentobarbital, phenadoxone, phenazocine, phencyclidine, phendimetrazine, phenmetrazine, pheneridine, piminodine, prodilidine, properidine, propoxyphene, propofol, cocaine, racemethorphan, racemorphan, racemoramide, remifentanil, secobarbital, sufentanil, talbutal, thebaine, thiamylal, thiopental, tramadol, trimeperidine, and vinbarbital.

In addition to the above, the following scheduled drugs or abused drugs may also be incorporated into the oral appliance, including, but not limited to, allobarbitone, alprazolam, eszopiclone, ramelteon, amylobarbitone, aprobarbital, barbital, barbitone, benzphetamine, brallobarbital, bromazepam, brotizolam, buspirone, butalbital, butobarbitone, butorphanol, camazepam, captodiame, carbromal, carfentanil, carpipramine, cathine, chloral, chloral betaine, chloral hydrate, chloralose, chlordiazepoxide, chlorhexadol, chlormethiazole edisylate, chlormezanone, cinolazepam, clobazam, potassium clorazepate, clotiazepam, cloxazolam, cyclobarbitone, delorazepam, dexfenfluramine, diazepam, diethylpropion, difebarbamate, difenoxin, dronabinol, enciprazine, estazolam, ethyl loflazepate, etizolam, febarbamate, fencamfamin, fenfluramine, fenproporex, fluanisone, fludiazepam, flunitraam, flunitrazepam, flurazepam, flutoprazepam, gepirone, glutethimide, halazepam, haloxazolam, hexobarbitone, ibomal, ipsapirone, ketamine, ketazolam, loprazolam mesylate, lorazepam, lormetazepam, mazindol, mebutamate, medazepam, mefenorex, mephobarbital, meprobamate, metaclazepam, methaqualone, methohexital, methylpentynol, methylphenobarbital, midazolam, milazolam, morphine, nimetazepam, nitrazepam, nordiazepam, oxazepam, oxazolam, paraldehyde, pemoline, pentabarbitone, pentazocine, pentobarbital, phencyclidine, phenobarbital, phendimetrazine, phenmetrazine, phenprobamate, phentermine, phenyacetone, pinazepam, pipradol, prazepam, proxibarbal, pseudoephedrine, ephedrine, epinephrine, anabolic steroids (e.g., testosterone, or syhtestic anabolic steroids) human growth hormone, quazepam, quinalbaritone, secobarbital, secbutobarbitone, sibutramine, temazepam, tetrazepam, triazolam, triclofos, zalepan, zaleplon, zolazepam, zolpidem, and zopiclone. Certain compounds described herein may exist in particular geometric or stereoisomeric forms.

In some embodiments, the medicament comprising chlorhexidine is in an amount of from about 0.001% w/w, v/v or w/v to about 5% w/w, v/v or w/v of the composition. In some embodiments, the medicament comprising chlorhexidine is in an amount of from about 0.001% w/w, v/v or w/v to about 20% w/w, v/v or w/v of the composition. In some embodiment, the medicament comprising chlorhexidine is in an amount from about 0.001, 0.005, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, about 20 w/w, v/v or w/v of the composition. In some embodiments, the chlorhexidine is chlorhexidine gluconate in an amount from about 0.0001% to about 5% w/w, v/v or w/v of the composition; from about 0.0001% to about 1% w/w, v/v or w/v of the composition; or from about 0.025% to about 1% w/w based on the total w/w of the composition. In some embodiments, the chlorhexidine is chlorhexidine gluconate in an amount of about 0.05% to about 0.06% w/w, v/v or w/v based on the total w/w, v/v or w/v of the composition.

Chlorhexidine is used for a variety of fields with different effective dosage ranges through its weight, and/or volume percentage in a composition. Chlorhexidine can be used as an active ingredient for treatment, can be used as a preservative for enhancing the stability of the composition, can be used as an antimicrobial against bacteria and other pathogens, or a combination thereof. Chlorhexidine can be used as an antiseptic and a disinfectant, which are effective against a wide variety of gram-positive and gram-negative bacteria, fungi, yeast and select viruses. Chlorhexidine has been used since 1959 and is widely available throughout the world. Chemically, chlorhexidine is a strong base and is most stable in its salt forms. Chlorhexidine gluconate (1,1′-hexamethylene bis [5-(p-chlorophenyl biguanide]di-D-gluconate), also known as chlorhexidine digluconate, is a salt formed from chlorhexidine and gluconic acid.

Chlorhexidine salts are adsorbed onto the cell walls of microorganisms, resulting in disruption of the cell wall integrity and leakage of intracellular contents. At low concentrations, chlorhexidine is a bacteriostatic agent, and at higher concentrations it becomes bactericidal. A primary benefit of chlorhexidine is its ability to kill bacteria on contact and remain non-toxic to mammalian cells.

Chlorhexidine salts are cationic, which facilitates their adsorption onto the surfaces of the oral mucosa, teeth and plaque, all of which have a net negative charge. The adsorbed chlorhexidine is gradually released from these tissues by diffusion. Thus, chlorhexidine has a substantial residual effect in that it retards microbial growth in the mouth for prolonged periods after application, allowing for either interval use or for daily application.

Chlorhexidine has been marketed for use in the oral cavity in many forms, including mouthwashes (usually 0.1-0.2%), 2% topical oral drops, lozenges, implantable chips, etc. In many countries, these preparations are sold over the counter. In the United States, chlorhexidine gluconate is available via prescription and over the counter (OTC).

In some embodiments, chlorhexidine is chlorhexidine digluconate (CHG), which is the gluconate salt form of chlorhexidine, a biguanide compound used as an antiseptic agent with topical antibacterial activity. Chlorhexidine digluconate is positively charged and reacts with the negatively charged microbial cell surface, thereby destroying the integrity of the cell membrane. Subsequently, chlorhexidine gluconate penetrates the cell and causes leakage of intracellular components leading to cell death. Since gram positive bacteria are more negatively charged, they are more sensitive to this agent. Chlorhexidine is largely used as a popular disinfectant in a range of healthcare products, including topical skin disinfectants, central venous catheters, hand washing solutions, and needleless IV connectors.

In the U.S., chlorhexidine for dental use is limited to prescription status and is available as an oral rinse and as a 2.5 mg chip (PerioChip®-Astra). The chip contains 2.5 mg of chlorhexidine gluconate in a glycerin and gelatin matrix and is indicated as an adjunct in scaling and root planning procedures for the reduction of a single pocket depth for each chip placed in patients with adult periodontitis (Drug Facts and Comparisons, 1999). Chlorhexidine gluconate rinse is available in the U.S. as a 0.12% solution (1.2 mg/ml) for the treatment of gingivitis. This commercial rinse is usually flavored with anise or mint and contains 11.6% (23 proof) alcohol by weight.

Any suitable source of chlorhexidine can be used in the compositions and methods of this disclosure. Suitable chlorhexidine starting materials include chlorhexidine salts, as they have enhanced stability over the parent chlorhexidine. In various embodiments described in this disclosure, chlorhexidine gluconate (also known as chlorhexidine digluconate), is a useful salt due to its high-water solubility. Other useful compounds include chlorhexidine diacetate and chlorhexidine dihydrochloride or a combination thereof. In some embodiments, the chlorhexidine used in the current application can be chlorhexidine digluconate 20% in water that can be obtained from Cyalume Specialty Products (Bound Brook, NJ, USA) as CAS number 18472-51-0.

The chlorhexidine in the composition can be used as a preservative in that when added to the hydrogel (e.g., PVA) it reduces microbial loads, suppresses their proliferation, and/or prevents new microbial contamination. In this way, the chlorhexidine maintains the integrity of the composition (e.g., hydrogel) and makes for a more stable product that is free of microbial contaminants.

The chlorhexidine in the composition can also function as an antimicrobial agent in that when added to the hydrogel (e.g., PVA) it kills microorganisms or stops or reduces their growth. In this way, the chlorhexidine can help reduce or prevent microbial growth in the area that it contacts in the oral cavity that can help wound healing. The chlorhexidine therefore can have multiple functions (e.g., preservative function, antimicrobial function and wound healing function) in the composition (e.g., hydrogel).

In various embodiments, the medicament (e.g., chlorhexidine) is useful as a suitable preservative, or antimicrobial for wound healing in the composition. The chlorhexidine comprises from about 0.01, 0.015, 0.02, 0.025 0.03, 0.035 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 1, 2, 3, 4, 5, 6, 7, 8, 9, 10, 11, 12, 13, 14, 15, 16, 17, 18, 19, to about 20% w/w, v/v or w/v of a carrier such as the hydrogel or the composition.

In various embodiments, the medicament such as chlorhexidine contacts teeth and/or gingival soft tissues from about 30 minutes to about 24 hours. In some embodiments, the medicament such as chlorhexidine contacts teeth and/or gingival soft tissues from about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 36, 48, 60 to about 72 hours. In various embodiments, the teeth and/or soft tissue areas of the oral cavity contacting the medicament such as chlorhexidine include a subgingival space, a gingival crevice, periodontal tissue, or a combination thereof. In some embodiments, the teeth and/or soft tissue areas of the oral cavity contacting the medicament such as chlorhexidine includes a wound in a subgingival space, a wound of the gingiva, a wound in a gingival crevice, a wound in a periodontal tissue, or a combination thereof.

In various embodiments, the chlorhexidine is delivered in an amount of 5.0 mg per day. In various embodiments, the chlorhexidine is delivered in an amount from about 0.5, 1, 1.5, 2, 2.5, 3, 3.5, 4, 4.5, 5, 5.5, to about 6.0 mg per day to the oral wound.

In various embodiments, the oral appliance may deliver medicament (e.g., chlorhexidine) as well as other components, including for example, mannose polysaccharides from aloe vera, xylitol and essential oils of cinnamon, clove and thyme and other excipients described in this disclosure. Generally, an “excipient” refers to an inert or inactive substance used in the production of pharmaceutical products. Useful excipients for the compositions of this disclosure include, without limitation, a surfactant, emollients, and/or enhancer. Non-limiting examples of excipients are described in Remington's Pharmaceutical Sciences by E. W. Martin, and include cellulose, starch, gelatin, sodium stearate, glycerol monostearate, sodium chloride, water, ethanol, or the like or combinations thereof. In some embodiments, the composition also contains pH buffering reagents, wetting or emulsifying agents. These agents are chosen that do not interfere with the medicament (e.g., chlorhexidine) preservative activity or antimicrobial activity and do not rot the teeth and/or gums. In some aspects, the composition can include an alcohol, and, in other aspects, the composition can be alcohol free.

Antioxidants such as vitamin E or coenzyme Q or a colorant in non-toxic concentration are also useful excipients that can be added to the hydrogel (e.g., PVA) or the composition of this disclosure.

In various embodiments, some areas of the non-porous polymer material such as, for example, ethylene vinyl acetate of the oral appliance do not contain the hydrogel and the non-porous polymer material may function to hold or lock a portion of the hydrogel in place so that the hydrogel (e.g., containing medicament or no medicament) can contact the appropriate target site. Thus, in some embodiments, the hydrogel is held in position at discrete regions of the oral appliance, for example, in a channel so that when the oral appliance is worn, the hydrogel will be adjacent to and contact the target tissue of the oral cavity.

The hydrogel can have the medicament (e.g., chlorhexidine) uniformly disposed in the hydrogel. For example, the interior surface of the oral appliance can have a channel and the hydrogel having the medicament (e.g., chlorhexidine) disposed uniformly within it can be disposed within that channel. The hydrogel having the medicament (e.g., chlorhexidine) forms a protective barrier between the wound and prevents or reduces further irritation and contamination of the oral wound. In some embodiments, the hydrogel forms a protective film that also allows the oral wound to heal.

In some embodiments, the hydrogel can have medicament (e.g., chlorhexidine) disposed at discrete regions of the hydrogel, where the hydrogel can then be disposed at discrete regions of the oral appliance. For example, the hydrogel can have a discrete region or discrete regions containing medicament, which then can be disposed at discrete regions of the oral appliance. In this way, specifically targeted medicament delivery to discrete regions of the oral cavity adjacent to the oral appliance can be controlled to limit or reduce unwanted off-target effects and provide additional comfort to the wearer even at regions of the oral cavity that do not require treatment as the hydrogel by itself, with or without medicament, provides a nice comfortable watery and absorbent cushion for the wearer of the oral appliance to experience.

In some embodiments, the medicament including the preservative, antimicrobial, or wound healing agent may be in powder, liquid, solid, solution, or suspension (e.g., hydrogel) form and disposed on or impregnated in the oral appliance. This may occur during manufacture of the oral appliance or it may occur after the oral appliance is made. For example, on the core polymer material of the oral appliance, the preservative, antimicrobial, or wound healing agent may be layered by solution or suspension layering or powder layering techniques. In solution or suspension layering, the preservative, antimicrobial, or wound healing agent and any inactive ingredients (excipients, binders, etc.) are suspended or dissolved in water or an organic solvent. The resulting liquid is sprayed onto the outside of the oral appliance to make the polymer material have the desired potency. Solution or suspension layering may be conducted using a wide variety of process techniques, for example, by fluidized bed, Wurster bottom spray techniques, or the like. When the desired potency has been achieved, the polymer material is dried to the desired residual moisture content. Powdered layering involves the application of a dry powder to the oral appliance. The powder may contain the preservative, antimicrobial, or wound healing agent, or may include excipients such as a binder, flow aid, inert filler, and the like. In the powder layering technique, a pharmaceutically acceptable liquid, which may be water, organic solvent, with or without a binder and/or excipient, is applied to the oral appliance while applying the dry powder until the desired potency is achieved. When the desired potency has been achieved, the oral appliance may be dried to the desired moisture content.

In various embodiments, the preservative, antimicrobial, or wound healing agent is in liquid form and is capable of diffusing through and within the oral appliance comprising a polymer material. In various embodiments, the liquid preservative, antimicrobial, or wound healing agent may flow or diffuse from one portion of the oral appliance to another portion. In some embodiments, the liquid preservative, antimicrobial, or wound healing agent may not flow or diffuse within the oral appliance. In some embodiments, the liquid preservative, antimicrobial, or wound healing agent is confined within the regions of the oral appliance corresponding to the treatment area. In some embodiments, the liquid preservative, antimicrobial, or wound healing agent is not capable of flowing or diffusing into the non-porous regions of the oral appliance.

In some embodiments, other preservatives, antimicrobials, or wound healing agents may be used either alone or in addition to the medicament (e.g., chlorhexidine) to treat infection or a wound, including without limitation, antiseptic agents, antibacterial agents; quinolones and in particular fluoroquinolones (e.g., norfloxacin, ciprofloxacin, lomefloxacin, ofloxacin, levofloxacin, etc.), aminoglycosides (e.g., gentamicin, tobramycin, etc.), glycopeptides (e.g., vancomycin, etc.), lincosamides (e.g., clindamycin), cephalosporins (e.g., first, second, third generation) and related beta-lactams, macrolides (e.g., azithromycin, erythromycin, etc.), nitroimidazoles (e.g., metronidazole), penicillins, polymyxins, tetracyclines, or combinations thereof.

The preservative, antimicrobial, or wound healing agents include antiseptics. Suitable antiseptics that can be in the hydrogel (e.g., PVA) include chlorhexidine, chlorhexidine gluconate, chlorhexidine digluconate, hexetidine, hydrogen peroxide, sodium hypochlorite, cetylpyridinium chloride, triclosan, methyl salicylate, povidone-iodine, alcohol, boric acid, iodine, hexachlorophene, or a combination thereof. Astringents, for example, zinc chloride are also useful excipients for the oral appliances described in this disclosure.

The amount of the preservative, antimicrobial, or wound healing agent contained within the oral appliance, will vary widely depending on the effective dosage required and rate of release from the polymer material and the length of the desired delivery interval. The dosage administered to the patient can be single or multiple doses and will vary depending upon a variety of factors, including the agent's pharmacokinetic properties, patient conditions and characteristics (sex, age, body weight, health, size, etc.), extent of symptoms, concurrent treatments, frequency of treatment and the effect desired. These factors can readily be determined by those of ordinary skill in the art.

In various embodiments, the polymer material of the oral appliance contains a preservative. In some embodiments, the polymer material of the oral appliance is designed to release an antimicrobial, or wound healing agent as a bolus dose of the antimicrobial, or wound healing agent, a single dose of the antimicrobial, or wound healing agent, or multiple doses of the antimicrobial, or wound healing agent all preloaded with a specific dosage or amount at the manufacturing facility.

In some embodiments, the amount of medicament such as preservative, antimicrobial, or wound healing agent, disposed in the hydrogel can be in an amount of from about 0.01% to about 20%, from about 0.1% to about 20%, from about 1% to about 20%, from about 1% to about 10% or from about 1% to 5% w/w, v/v or w/v based on the weight of the oral appliance. The amount of medicament (e.g., preservative, antimicrobial, or wound healing agent) disposed in the hydrogel can be in an amount of from about 0.01, 0.015, 0.02, 0.025 0.03, 0.035 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 1, 2, 3, 4, 5, 6, 7, 8, 9, 10, 11, 12, 13, 14, 15, 16, 17, 18, 19, to about 20% based on the weight of the hydrogel, composition or oral appliance. For example, a first oral appliance can be made specific or custom fit for a patient. The oral appliance can have porous and non-porous regions, the antimicrobial can be in the porous and/or non-porous regions, for example, from about 0.01% to about 5% w/w, v/v or w/v chlorhexidine based on the total weight of the oral appliance or hydrogel. A second oral appliance can be made for that patient with the same or a lesser amount of preservative, antimicrobial, or wound healing agent, for example 2.5% or 0.00.5% chlorhexidine based on the total weight of the hydrogel, composition or oral appliance.

The amount of the medicament including preservative, antimicrobial, or wound healing agent contained within the oral appliance, will vary widely depending on the effective dosage required and rate of release from the polymer material, the length of the desired delivery interval and the surface area to be covered by the medicament/hydrogel. The dosage administered to the patient can be single or multiple doses and will vary depending upon a variety of factors, including the agent's pharmacokinetic properties, patient conditions and characteristics (sex, age, body weight, health, size, etc.), extent of symptoms, concurrent treatments, frequency of treatment and the effect desired. These factors can readily be determined by those of ordinary skill in the art.

In some embodiments, the medicament including the preservative, antimicrobial, or wound healing agent (e.g., chlorhexidine) can be disposed in the hydrogel in an amount of about 0.00001, 0.00005, 0.00010, 0.00015, 0.00020, 0.00025, 0.00030, 0.00035, 0.00040, 0.00045, 0.00050, 0.00055, 0.00060, 0.00065, 0.00070, 0.00075, 0.00080, 0.00085, 0.00090, 0.00095, 0.0010, 0.0015, 0.0020, 0.0025, 0.0030, 0.0035, 0.0040, 0.0045, 0.0050, 0.0055, 0.0060, 0.0065, 0.0070, 0.0075, 0.0080, 0.0085, 0.0090, 0.0095, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5 to about 5% w/v, w/w and/or v/v based on the total w/v, w/w and/or v/v of the hydrogel.

In some embodiments, the composition can be disposed anywhere in or on the interior or exterior surface of the oral appliance adjacent to the gum and/or other soft tissue areas of the oral cavity including the front, back, occlusal surfaces of one or more teeth. Some portions of teeth that do not require the composition are sealed with the non-porous material which can be a coating, cross-linked with a porosity reducing agent or comprising non-porous material such that the composition cannot penetrate said portions. In some embodiments, the composition may be disposed in or may enter the non-porous region. However, the composition disposed in the non-porous region will not release the preservative, antimicrobial, or wound healing agent or will release the preservative, antimicrobial, or wound healing agent at a reduced rate.

In some embodiments, the preservative, antimicrobial, or wound healing agent may enter or flow into the non-porous regions, but the preservative, antimicrobial, or wound healing agent will be released more slowly from these regions. For example, the hydrogel having the preservative, antimicrobial, or wound healing agent can be disposed at discrete non-porous regions adjacent to the treatment area or uniformly disposed throughout the device. In this example, the preservative, antimicrobial, or wound healing agent will not be released to other regions that do not correspond to the treatment area.

As the interior and/or exterior surface of the oral appliance contacts the oral cavity, the preservative, antimicrobial, or wound healing agent is released from the polymer such that all or parts of the oral appliance will degrade over time by the action of enzymes, by hydrolytic action and/or by other similar mechanisms in the oral cavity. In various embodiments, the degradation can occur either at the surface of the oral appliance at discrete positions (heterogeneous or surface erosion) or uniformly throughout the oral appliance (homogeneous or bulk erosion). In some embodiments, all or discrete portions of the interior surface will degrade, particularly those regions containing the hydrogel having the preservative, antimicrobial, or wound healing agent, and release the preservative, antimicrobial, or wound healing agent at or near the target site in the oral cavity. The oral appliance will cover at least a portion of the teeth and/or gums, by applying the device over the axis to cover the area of the teeth and/or gums, and the oral appliance will be adjacent to the gingival sulcus or other soft tissue or hard tissue areas, which will allow the preservative, antimicrobial, or wound healing agent, if desired, to be released from the polymer to these areas.

### Hydrogel

The oral appliance of the present application comprises a hydrogel loaded in the interior, exterior or both the interior and exterior of the oral appliance. In some embodiments, the hydrogel is a network of polymer chains that are hydrophilic but water insoluble. Hydrogels are sometimes found as colloidal gels in which water is the dispersion medium. The hydrogel can include natural polymers, synthetic polymers, or a combination thereof. Natural polymers include, but are not limited to, chitosan, sodium alginate, hyaluronic acid, collagen, gelatin, chondroitin sulfate, silk protein or a combination thereof. Synthetic polymers, include, but are not limited to, polyethylene glycol (PEG), polyvinyl alcohol (PVA), poly(lactic-co-glycolic acid), ethylene glycol dimethacrylate (EGDMA), hydroxypropylcellulose (HPC) or poly(2-hydroxyethyl methacrylate) (pHEMA) or a combination thereof.

PVA is a water-soluble polymer hydrolyzed from polyvinyl acetate with good biocompatibility, a high modulus of elasticity, and easily adjustable physical properties. In some embodiments, the incorporation of PVA and natural polymers in the hydrogel compensates for the poor mechanical properties of natural polymer-based hydrogels and still allows favorable cytocompatibility and bioactivity. In some embodiments, the PVA can be obtained from Sigma Aldrich (Saint Louis, MO, USA) in powdered form as CAS number 9002-89-5.

In some embodiments, the polymer content of the hydrogel is in an amount of about 5% w/w, v/v or w/v to about 99% w/w, v/v or w/v of the composition or about 50% w/w, v/v, or w/v of the composition. In some embodiments, the polymer content of the hydrogel is in an amount from about 61% to about 93% w/w, v/v or w/v of the composition. In some embodiments, the polymer content (PVA) of the hydrogel or composition is in an amount of from about 5% w/w, v/v or w/v, 6, 7, 8, 9, 10, 11, 12, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 91, 92, 93, 94, 95, 96, 97, 98, to about 99% w/w, v/v or w/v of the hydrogel or composition. In some embodiments, the hydrogel comprises the polymer polyvinyl alcohol (PVA) and water. In some embodiments, the PVA is in an amount from about 12% to about 19% w/w, v/v or w/v of the composition. In some embodiments, the PVA is in an amount from about 12, % w/w, v/v or w/v, 12.5, 13, 13.5, 14, 14.5, 15, 15.5, 16, 16.5, 17, 17.5, 18, 18.5, to about 19% w/w, v/v or w/v of the composition. In some embodiments, the water in the composition or in the hydrogel is in an amount from about 50% to about 82% w/w, v/v or w/v of the composition. In some embodiments, the water is from about 50%, 50.5, 51, 51.5, 52, 52.5, 53, 53.5, 54, 54.5, 55, 55.5, 56.5, 57, 57.5, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5 to about 82% w/w, v/v or w/v of the composition. The water can be sterile water, distilled water, de-ionized water, tap water, or a combination thereof. For example, in some embodiments, the water can be distilled water.

The water that is in the composition or polymer can be, for example, from about 65% w/w, w/v, or v/v, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or to about 90% w/w, w/v, or v/v based on the total weight of the composition or polymer.

In various embodiments, the water content in the composition or hydrogel, after the polymer (e.g., PVA) is crosslinked can be from about 60% to about 75% w/w, v/v or w/v of the composition.

In various embodiments, the molecular weight of the polymer can be varied as desired. The choice of method to vary molecular weight is typically determined by the composition of the gel (e.g., polymer, versus non-polymer).

In various embodiments, when the polymer is crosslinked by UV curing, a crosslinking agent can be used. Nonlimiting examples of commercially available crosslinking agents include, for example, diethoxyacetophenone (DEAP), dimethoxyphenylacetophenone (Irgacure 651), benzoylcyclohexanol (Irgacure 184), or hydroxydimethylacetophenone (Darocure 1173). In some embodiments, the amount of crosslinking agent can vary from about 0.015, 0.020, 0.025, 0.030, 0.035, 0.040, 0.045, 0.050, 0.055, 0.060, 0.065, 0.070, to about 0.075 mL.

Suitable hydrogel polymers may be soluble in an organic solvent. The solubility of a polymer in a solvent varies depending on the crystallinity, hydrophobicity, hydrogen-bonding and molecular weight of the polymer. Lower molecular weight polymers will normally dissolve more readily in an organic solvent than high-molecular weight polymers. A polymeric hydrogel that includes a high molecular weight polymer tends to coagulate or solidify more quickly than a polymeric composition that includes a low-molecular weight polymer. Polymeric hydrogel formulations that include high molecular weight polymers also tend to have a higher solution viscosity than a polymeric hydrogel that includes low-molecular weight polymers. In various embodiments, the molecular weight of the hydrogel polymer can be a wide range of values. The average molecular weight of the hydrogel polymer can be from about 1000 to about 10,000,000; or about 1,000 to about 1,000,000; or about 5,000 to about 500,000; or about 10,000 to about 100,000; or about 20,000 to 50,000 g/mol; or about 300 kDa to about 1,000 kDa.

In various embodiments, the hydrogel has an inherent viscosity (abbreviated as “I.V.” and units are in deciliters/gram), which is a measure of the hydrogel's molecular weight and degradation time (e.g., a hydrogel with a high inherent viscosity has a higher molecular weight and may have a longer degradation time). Typically, when the polymers have similar components but different molecular weights, a hydrogel with a high molecular weight provides a stronger matrix and the matrix takes more time to degrade. In contrast, a hydrogel with a low molecular weight degrades more quickly and provides a softer matrix. In various embodiments, the hydrogel has a molecular weight, as shown by the inherent viscosity, from about 0.10 dL/g to about 1.2 dL/g or from about 0.10 dL/g to about 0.40 dL/g. Other IV ranges include but are not limited to about 0.05 to about 0.15 dL/g, about 0.10 to about 0.20 dL/g, about 0.15 to about 0.25 dL/g, about 0.20 to about 0.30 dL/g, about 0.25 to about 0.35 dL/g, about 0.30 to about 0.35 dL/g, about 0.35 to about 0.45 dL/g, about 0.40 to about 0.45 dL/g, about 0.45 to about 0.55 dL/g, about 0.50 to about 0.70 dL/g, about 0.60 to about 0.80 dL/g, about 0.70 to about 0.90 dL/g, about 0.80 to about 1.00 dL/g, about 0.90 to about 1.10 dL/g, about 1.0 to about 1.2 dL/g, about 1.1 to about 1.3 dL/g, about 1.2 to about 1.4 dL/g, about 1.3 to about 1.5 dL/g, about 1.4 to about 1.6 dL/g, about 1.5 to about 1.7 dL/g, about 1.6 to about 1.8 dL/g, about 1.7 to about 1.9 dL/g, and about 1.8 to about 2.1 dL/g.

In some embodiments, when the polymer materials have different chemistries (e.g., high MW DLG 5050 and low MW DL), the high MW polymer may degrade faster than the low MW polymer.

In various embodiments, the hydrogel can have a viscosity of about 300 to about 5,000 centipoise (cp). In other embodiments, the hydrogel can have a viscosity of from about 5 to about 300 cps, from about 10 cps to about 50 cps, or from about 15 cps to about 75 cps at room temperature. The hydrogel may optionally have a viscosity enhancing agent such as, for example, hydroxypropyl hydroxypropyl methylcellulose, hydroxyethyl methylcellulose, carboxymethylcellulose and salts thereof, Carbopol, poly-(hydroxyethylmethacrylate), poly-(methoxyethylmethacrylate), poly(methoxyethoxyethyl methacrylate), polymethylmethacrylate (PMMA), methylmethacrylate (MMA), gelatin, polyvinyl alcohols, propylene glycol, mPEG, PEG 200, PEG 300, PEG 400, PEG 500, PEG 600, PEG 700, PEG 800, PEG 900, PEG 1000, PEG 1450, PEG 3350, PEG 4500, PEG 8000 or combinations thereof. In some embodiments, the composition or the hydrogel has a viscosity from about 5000 cP to about 5300 cP. In some embodiments, the composition or the hydrogel has a viscosity from about 4000 cP to about 6600 cP. In some embodiments, the composition or the hydrogel has a viscosity from about 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500 to about 6600 cP. In some embodiments, the viscosity can be maintained at a temperature from about 15, 16, 17, 18, 19, 20, 21, 22, 23, 24, 25, 26, 27, 28, 29 to about 30 degrees Celsius. In some embodiments, the composition has a viscosity from about 5000 cP to about 5300 cP at 25 degrees Celsius.

In various embodiments, the hydrogel is made of high molecular weight biocompatible elastomeric polymers of synthetic or natural origin. In other embodiments, the hydrogel material can hold collected biological materials when the hydrogel material is hypo-saturated, saturated, or supersaturated. There are many advantages resulting from using a hydrogel as the carrier for the medicament (e.g., antimicrobial, preservative, or wound healing agent, etc.) used in oral appliances described herein. Generally, hydrogel materials provide an effective contact medium for gum compression and for collecting biological materials for diagnosis. The above can hold the sample (e.g., saliva, blood, cells, various oral fluids, inflammatory factors, other biologic markers, etc.) when the oral appliance is removed and then the oral appliance can be sent to the lab for testing. Sending out the entire oral appliance to the lab can prevent cross contamination by the patient's hands contaminating the sample collected by the hydrogel. However, in another way, in some embodiments, only the hydrogel can be removed and then sent out to the lab for testing.

In various embodiments, the hydrogel is formed by physical crosslinking of polyvinyl alcohol (PVA) solution. PVA consists of a secondary alcohol group attached to a linear carbon chain. The alcohol group allows for hydrogen bonding and, therefore, PVA dissolved in aqueous solution can produce a hydrogel with high water content. However, such hydrogels have poor mechanical properties due to the low gelation density. In order to prepare PVA hydrogels that can maintain their shape and location in the oral appliance while maintaining a high water content further PVA crosslinking is required. In some embodiments, PVA is crosslinked using chemical agents such as formaldehyde and glutaraldehyde to form a non-resorbable foam. In some embodiments, PVA is crosslinked through physical crosslinking methods without the use of additional chemicals, eliminating the risk of leaving residual amounts of toxic crosslinking agents. Physical crosslinking can be accomplished using a freeze-thaw (FT) cycling method in which a solution of PVA is allowed to undergo repeated freezing and thawing cycles. Thermal cycling leads to physical crosslinking via formation of structured crystalline domains of the polymer chains through phase separation. In FT crosslinking, first the hydrogel is brought down to a temperature of −20° C. to freeze the water phase. This creates regions of high polymer concentration, where crystallites are formed, as well as regions of low polymer concentration resulting in pores. The solution is then thawed back to room temperature leading to the formation of a solid hydrogel.

In some embodiments, after the composition or polymer (e.g., PVA) is loaded in the oral appliance, it can be subjected to freezing at a temperature of about −20° C. and then thawed at room temperature or a temperature of about 20° C., which allows crosslinking of the composition or polymer (e.g., PVA). This can be performed, for example, in three cycles.

### Humectant

The hydrogel composition of the current application includes a humectant. In some embodiments, the humectant is in an amount of about 0.5% w/w, v/v or w/v to about 15% w/w, v/v or w/v based on a total w/w, v/v or w/v of the composition. In some embodiments, the humectant is in an amount from about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5 to about 15% w/w, v/v or w/v based on a total w/w, v/v or w/v of the composition.

Glycerol (C₃H₈O₃), also known as glycerin, is an odorless, colorless, oily, viscous liquid that has a sweet taste. Glycerol acts as humectant in the composition, helping the hydrogel preserve moisture and water content during storage. In some embodiments, the glycerol can be obtained from Sigma Aldrich (Saint Louis, MO, USA) in liquid form as CAS number 56-81-5.

In some embodiments, glycerol is a humectant that is added to the hydrogel in the composition. In some embodiments, other humectants may be used either alone or in addition to glycerol, such as, polyethylene glycol, propylene glycol, hexylene glycol, butylene glycol, glyceryl triacetate, neoagarobiose, sugar alcohols (sugar polyols) such as sorbitol, xylitol, maltitol, polymeric polyols such as polydextrose, quillaia, urea, aloe vera gel, MP diol, alpha hydroxy acids such as lactic acid, honey and/or lithium chloride. In some embodiments, the humectant can be a sterile fluid, such as sterile water, distilled water, double distilled water, de-ionized water, water, NaCl (saline e.g., 0.90 saline or 0.45 saline), D5W (dextrose in 5% water), D5NS (dextrose in 5% water and normal saline) and D5W/1/2NS (D5W and ½ normal saline) or mixtures thereof.

### Oral Appliance

Numerous custom fit oral appliances can be made in a variety of ways including by traditional thermoforming, 3D printing or additive manufacturing, or injection molding or other ways. Unlike orthodontic appliances, the present oral appliance is not designed to move teeth and is not an orthodontic appliance. Therefore, a plurality of oral appliances will be configured to fit the teeth in the same position as was imaged within the oral appliance. The teeth position will not change. However, the antimicrobial disposed in or on the oral appliance will be in the same or different areas at different stages of the treatment regimen with a variety of oral appliances. Thus, kits containing a plurality of oral appliances can be provided with different treatment plans. For example, as the patient condition improves, each oral appliance will have a decreasing amount of antimicrobial or the antimicrobial can change as the treatment progresses.

In various embodiments, the oral appliance is monolithic or a single piece and the interior surface custom fit and formed to fit contours of the teeth and/or soft tissue areas inside the oral cavity of a patient in need of treatment. In this embodiment, the antimicrobial including the polymer is disposed at discrete regions of the oral appliance. The preservative, antimicrobial, or wound healing agent is part of the device and in some embodiments, the preservative, antimicrobial, or wound healing agent is not removable from it except by diffusion in the mouth. In certain embodiments, the oral appliance comprises, consists essentially of or consists of one, two, three, four, five or more oral appliances.

In some embodiments, the oral appliance is a single use, nonsterile, patient specific device, provided by a dental professional and worn by the patient following occurrence of an oral wound. In some embodiments, the oral appliance comprises at least one dental tray or consists of two patient specific dental trays, one for the lower jaw and one for the upper jaw. Each of the trays are designed with an integral channel along the location of the wound. In some embodiments, the channel is 3 mm in width and 2 mm in depth. In some embodiments, the channel has a width from about 0.5, 1, 1.5, 2, 2.5, 3, 3.5, 4, 4.5, 5, 5.5 to about 6 mm. In various embodiments, the channel has a depth from about 0.5, 1, 1.5, 2, 2.5, 3, 3.5, to about 4 mm. The length of the channel is determined by the size of the wound. The channel is pre-filled with the composition as described herein. As the length and therefore the total volume of the channel varies among individual patients, the amount of hydrogel in the channel is designed to remain constant over the surface area of the channel. Therefore, all tissue regardless of the tray size and wound length will be in contact with the same amount of hydrogel. In some embodiments, the hydrogel contains various amounts of effective doses of medicament (e.g., chlorhexidine) depending on the treatment dosing regimen. Once worn by the patient, the oral appliance creates a tight fit, gently pressing the hydrogel against the wound, keeping it moist and absorbing wound exudate, thus assisting wound healing.

The preformed channels on the oral appliance prevent the hydrogel from spreading throughout the oral cavity and inadvertent swallowing. Filling the preformed channels with hydrogel and the subsequent swelling of the hydrogel provides a gentle pressure on the wound which can further promote wound healing. In some embodiments, the hydrogel swells as the hydrogel absorbs additional fluid from other liquids after the delivery of the composition into an oral cavity. The hydrogel absorbs waste, exudate, saliva and other liquids present in the oral cavity. In some embodiments, the composition or the hydrogel swells from about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50% w/w of the composition before contacting the oral cavity.

In various embodiments, the hydrogel component of the oral appliance serves as mechanical barrier protecting the wound from environmental irritation and contamination and reduces pain while improving healing by providing a moist environment and absorbing the wound exudate. The channel when provided in the oral appliance maintains the hydrogel in place, and the medicament (e.g., chlorhexidine) in the hydrogel acts as preservative, antimicrobial or wound healing agent, preventing bacterial and fungal growth within the moist, nonsterile hydrogel during storage.

In various embodiments, the oral appliance can be made of a non-porous thermoplastic material. For example, the oral appliance can be made from vacuum thermoforming of ethylene-vinyl acetate (EVA), which is available from Keystone Industries (Myerstown, PA, USA) as CAS number 108-05-4. EVA is a thermoplastic copolymer of ethylene and vinyl acetate. For dental use, it is sourced in the form of rigid or flexible flat plates, in thicknesses of 1-5 mm, without the presence of blowing agents. In some embodiments, the thickness ranges from about 0.5, 1, 1.5, 2, 2.5, 3, 3.5, 4, 4.5, 5, 5.5, 6, 6.5, 7 to about 7.5 mm. In some embodiments, the EVA used is in form of 5 inch round sheets in 1 mm thickness.

In various embodiments, the oral appliance is not monolithic or a single piece. The medicament (e.g., preservative, antimicrobial, or wound healing agent) is disposed in a hydrogel, which is disposed in the interior or inside the oral appliance, but as a separate component to the oral appliance. For example, the medicament (e.g., preservative, antimicrobial, or wound healing agent) can be disposed in hydrogel that is configured to allow release of the medicament when the oral appliance is worn.

In some embodiments, oral appliances include, but are not limited to, oral trays, oral holders, oral covers, or the like that are designed to be placed within the oral cavity. The interior surface and/or exterior surface of the oral appliance contains the hydrogel having the medicament (e.g., preservative, antimicrobial, or wound healing agent) disposed anywhere within or on the oral appliance as part of the device. In some embodiments, the exterior surface of the oral appliance has the hydrogel containing the medicament (e.g., preservative, antimicrobial, or wound healing agent) and allows the medicament to be released to adjacent teeth and/or soft or hard tissue, or into the mouth in general.

In various embodiments, the oral appliance has a shelf life from about 1, 2, 3, 4, 5, 6, 7, 8, 9, 10, 11 to about 12 months. In some embodiments, the oral appliance has a shelf life of 3 months. Although the shelf life conditions may vary, in various embodiments, the oral appliance has a shelf life of 3 months when stored at temperatures from 25° C. to 60° C. and 50% to about 75% relative humidity. In some embodiments, the oral appliance with the composition is stored at temperatures from about 20, 25, 30, 35, 40, 45, 50, 55 to about 60° C. In some embodiments, the oral appliance with the composition is stored at a relative humidity from about 40, 45, 50, 55, 60, 65, 70 to about 75% relative humidity.

Numerous different oral appliances can be made by the methods of the present application, including custom fit oral appliances that correspond to a digital scan taken from the patient's mouth or impression molds. Custom fit oral appliances are generally described in U.S. Pat. No. 6,626,669 filed Apr. 15, 2002; U.S. Pat. No. 9,579,178 filed Jul. 12, 2013 and U.S. Pat. No. 9,649,182, to Peter J. Zegarelli, filed Jun. 18, 2015, as well as PCT/US2020/061500 filed on Nov. 20, 2020. The entire disclosure of these references are herein incorporated by reference into the present disclosure.

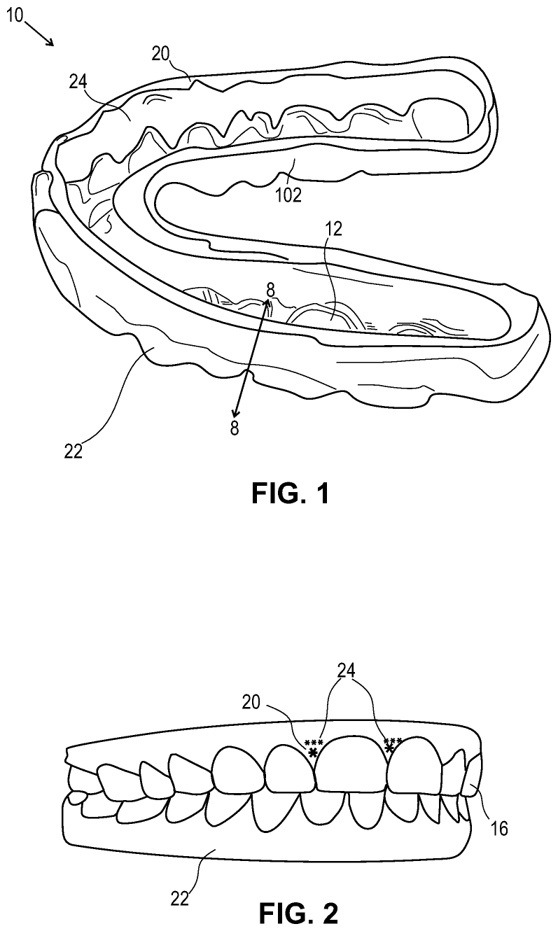

FIG. 1 is an enlarged side view of an embodiment of the oral appliance 10 before it is worn. The oral appliance is custom fit and has an interior surface 12 configured to contour at least a portion of the teeth and/or soft tissue areas of the oral cavity. The oral appliance also has an exterior surface 22. The interior surface, the exterior surface or both surfaces can comprise a hydrogel 24 that has medicament or no medicament. The interior surface 12 is configured to contact at least all or a portion of one or more teeth and/or soft tissue areas of the oral cavity in a patient. In this view the interior surface 12 is configured to contact the teeth and soft tissue. The interior surface 12 has a channel 102. The channel is configured to receive and secure the hydrogel, which can contain the medicament (e.g., chlorhexidine).

Oral appliances include, but are not limited to, oral trays, oral holders, oral covers, or the like that are designed to be placed within the oral cavity. The interior surface 12 and/or exterior surface 22 of the oral appliance can contain the hydrogel, which can contain the medicament (e.g., chlorhexidine). The hydrogel can be disposed anywhere within or on the oral appliance. For example, the hydrogel containing medicament (e.g., chlorhexidine) can be disposed at discrete regions adjacent to the treatment area (e.g., gingival sulcus 20) or uniformly disposed throughout the device. As the interior and/or exterior surface of the oral appliance contacts the oral cavity, medicament is released from the hydrogel by all or parts of the oral appliance contacting the desired treatment site or pressure from the device contacting tissue or fluid at the treatment site (e.g., gums, tissue, teeth, etc.).

It will be understood that the medicament (e.g., chlorhexidine) disposed in the hydrogel can function as a preservative to keep the hydrogel stable and reduce or inhibit growth of microorganisms. In other embodiments, the medicament (e.g., chlorhexidine) disposed in the hydrogel can function as an antimicrobial, where it is released and reduces microbial growth at the target tissue. In some embodiments, the medicament in the hydrogel can function both as a preservative and as an antimicrobial at the same or different concentrations in the hydrogel.

For example, the hydrogel containing the medicament (e.g., chlorhexidine) can be minimally released from the oral appliance. In this embodiment, the medicament (e.g., chlorhexidine) is in the hydrogel composition as a preservative and used to provide stability to the hydrogel and prevent or reduce microbial growth. As the hydrogel contacts the target tissue site and is emptied, fluids are wicked up and may be absorbed by the hydrogel in a fluid exchange, removing this contaminated exudate from the oral cavity.

In some embodiments, the hydrogel containing the medicament (e.g., chlorhexidine) can be released from the oral appliance. In this embodiment, the medicament (e.g., chlorhexidine) is in the hydrogel composition as an antimicrobial agent that reduces or prevents microbial growth at the site it is released to. For example, GCFs including exudate fluids containing bacteria, dead cellular structures, interstitial fluids, inflammatory factors, etc. can be removed from the target tissue site (e.g., oral soft and/or hard tissue) that allows healing of the oral tissue. In periodontal disease, for example, the greater the degree of inflammation, the greater the rate of flow of the GCF. Removing the GCF from the periodontal pocket creates space for the antimicrobial to occupy to inhibit or reduce microbial growth.

In some embodiments, the hydrogel and/or medicament in the hydrogel can degrade over time by the action of enzymes, by hydrolytic action and/or by other similar mechanisms in the oral cavity. In some embodiments, all or discrete portions of the hydrogel containing the medicament (e.g., a preservative, antimicrobial, or wound healing agent) will degrade and release the medicament at or near the target tissue site in the oral cavity. The oral appliance will cover at least a portion of the teeth and/or gums by applying the device over axis 8-8 to cover the area of the teeth and/or gums, and the oral appliance will be adjacent to the gingival sulcus region 20, where the medicament (e.g., a preservative, antimicrobial, or wound healing agent), if desired, will be released from the hydrogel to this area.

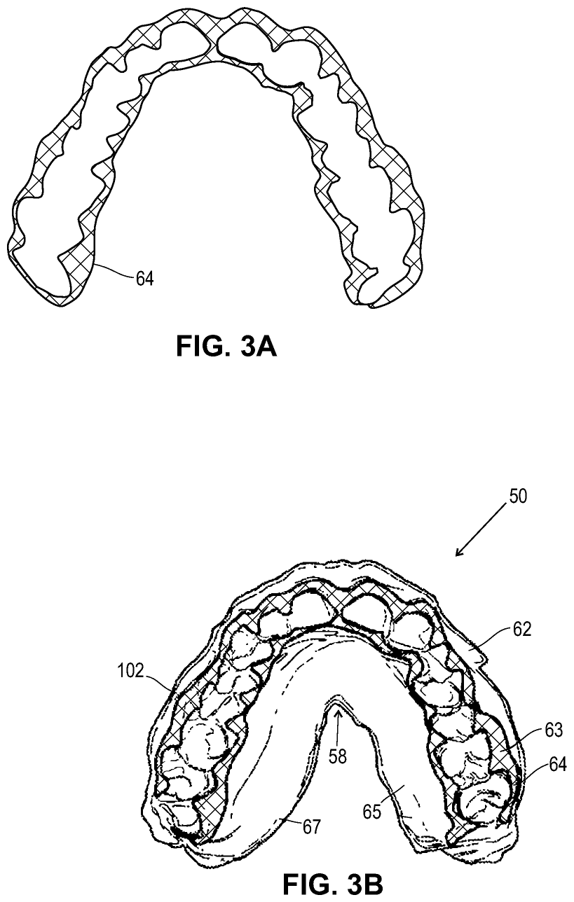

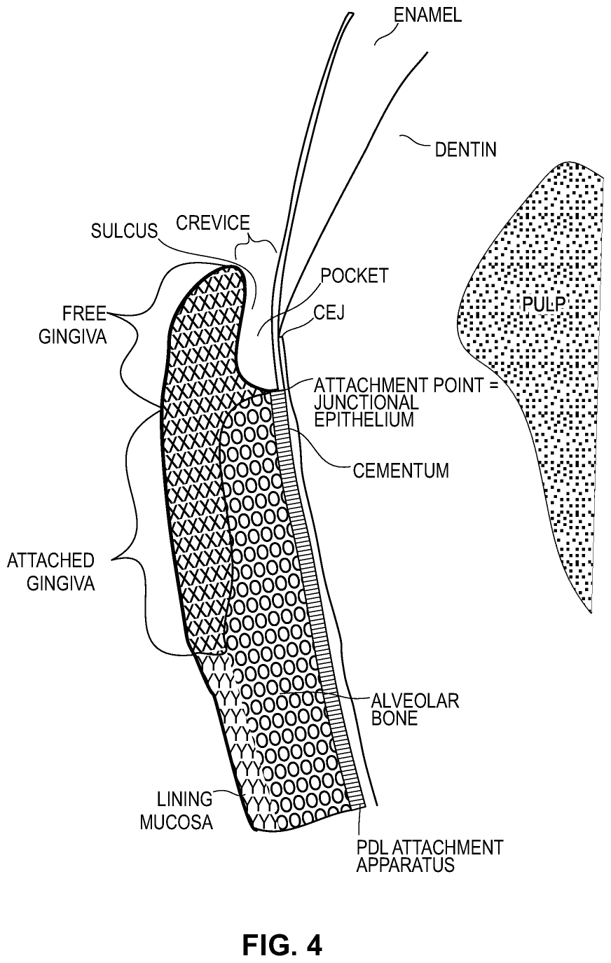

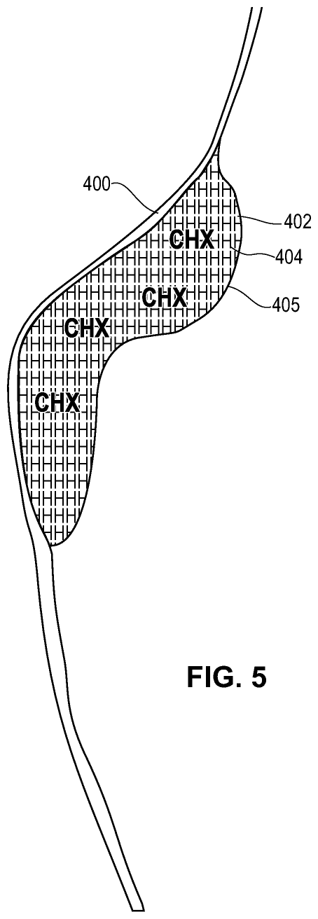

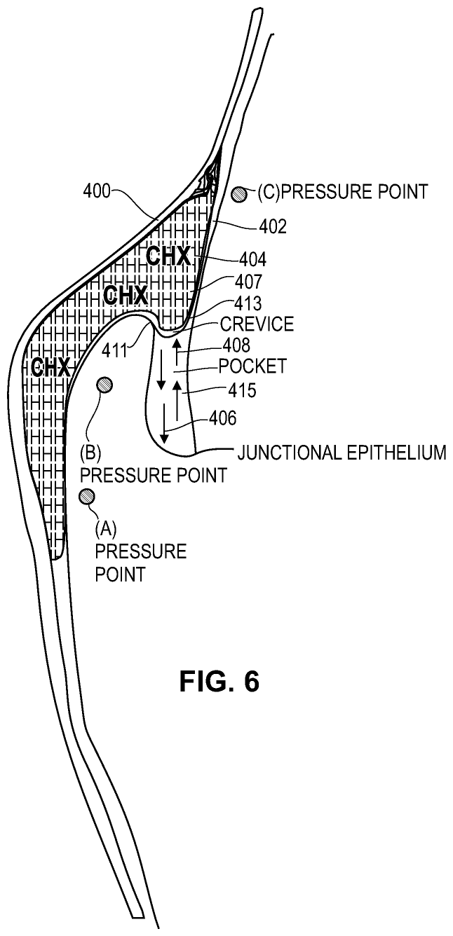

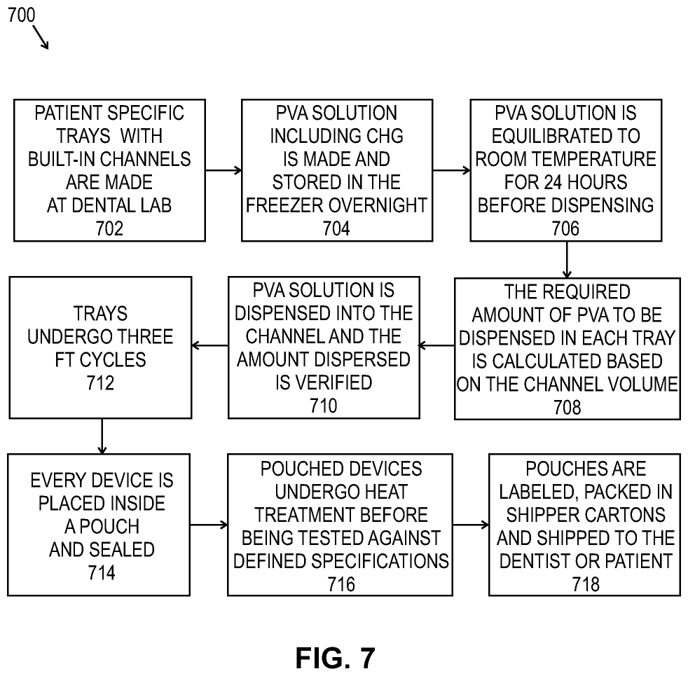

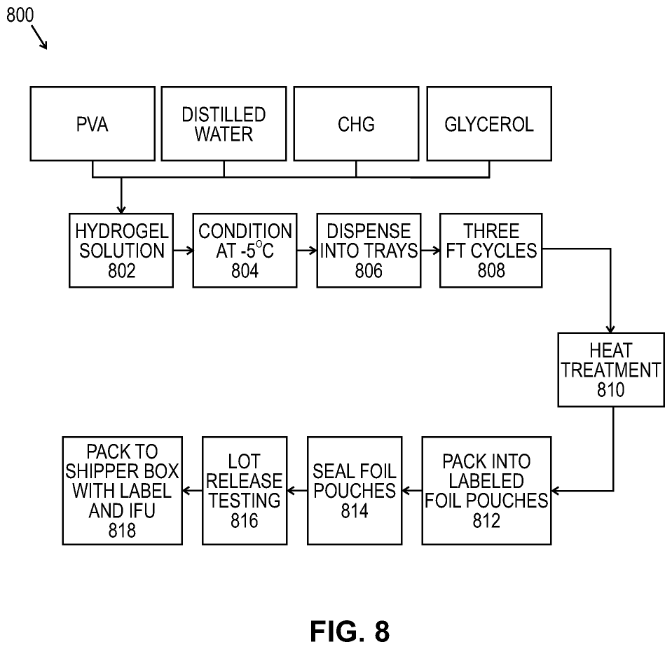

FIG. 2 illustrates an enlarged side view of an embodiment of the oral appliance, where a hydrogel 24 containing the medicament (e.g., a preservative, antimicrobial, or wound healing agent) is shown disposed in the hydrogel (e.g., PVA) that is adjacent to an infected tissue, which is shown as the gingival sulcus region 20. This view has the teeth 16 and gums loaded in the interior of the oral appliance and the medicament (e.g., a preservative, antimicrobial, or wound healing agent) is disposed in a polymer at a discrete region adjacent to the infected tissue of the oral cavity. The exterior surface 22 of the oral appliance can be transparent or non-transparent. It will be understood by those of ordinary skill in the art that the medicament (e.g., a preservative, antimicrobial, or wound healing agent) can be disposed uniformly throughout the hydrogel (e.g., PVA) or at a discrete region of the hydrogel (e.g., PVA) depending on the location of the treatment area.

FIG. 3A illustrates an enlarged view of a hydrogel 64 (e.g., cross linked hydrogel) that can be loaded on the exterior surface, interior surface, or both the interior surface and the exterior surface of the oral appliance. The hydrogel can have a medicament (e.g., chlorhexidine) disposed uniformly throughout the hydrogel or, in some embodiments, it can be disposed at a discrete region of the hydrogel and/or discrete regions of the oral appliance. The medicament can be for example, a preservative, an antimicrobial, a wound healing agent or a combination thereof. In some embodiments, the hydrogel can contain no medicament or preservative. The hydrogel once loaded into the oral appliance at discrete regions of it will contact discrete regions of the oral tissue (e.g., soft tissue and/or hard tissue) that contact the hydrogel and/or the oral appliance where treatment is desired.

FIG. 3B illustrates an enlarged view of an oral appliance 50 where the exterior surface is made from non-porous material. The oral appliance has a hydrogel 64 disposed at discrete regions 62 of the oral appliance. Shown is the hydrogel disposed in the interior surface of the oral appliance in a channel 102. The hydrogel can have a medicament (e.g., chlorhexidine) disposed uniformly throughout the hydrogel or, in some embodiments, the medicament can be disposed at discrete regions of the actual hydrogel and/or discrete regions of the oral appliance. The channel 102 loaded with the hydrogel containing medicament is disposed at a discrete region of the oral appliance that can be adjacent to and contact the gingival sulcus (gumline) 63 to allow treatment of infected tissue at the sulcus. It will be understood by those skilled in the art that, in some embodiments, that the hydrogel can contain no medicament but can comprise the polymer (e.g., PVA) and optionally a humectant.

The lower portion of the oral appliance corresponds to and will contact portions of the tongue and hard palate 65 as well as the soft palate 67 along each side of the oral cavity. The interior portion 58 of the oral appliance corresponds to and will contact portions of the dorsum of the tongue when pressed against it and the hard palate 65. The soft palate is shown as 67. One or more of these areas or other areas of the interior and/or exterior surface of the oral appliance can contain hydrogel.

FIG. 4 illustrates an enlarged cross-sectional view of the anatomy of the gums and a tooth including free gingiva, attached gingiva, lining mucosa, the alveolar bone, the periodontal pocket or crevice, the cementoenamel junction (CEJ), periodontal ligament, cementum, the enamel, dentin, pulp and the junctional epithelium. In some embodiments, at the beginning of treatment, the top portion of the periodontal pocket is targeted for delivery of the medicament (e.g., preservative, antimicrobial, or wound healing agent) of the composition in a top down approach. The top down approach is generally described in WO 2021/092405 filed Nov. 6, 2020 and published on May 14, 2021, which is assigned to Emanate Biomedical Inc. This entire disclosure is herein incorporated by reference.

In some embodiments, the oral appliance contains a carrier, which is a porous material (e.g., a hydrogel), which can contain the medicament (e.g., a preservative, antimicrobial, or wound healing agent (e.g., chlorhexidine)), which is released for a sustained period of time until the infection is reduced and is brought under greater control. The oral appliance described herein serves multiple purposes. It holds the preservative, antimicrobial, or wound healing agent in place with no or limited dilution by saliva or contamination by oral liquids, and it keeps antimicrobial at the top of the pocket. As the hydrogel is squeezed when the oral appliance is worn, the preservative, antimicrobial, or wound healing agent diffuses into the top of the pocket. The oral appliance design, characterized by the hydrogel placement over the gingival crevice, is akin to an encapsulation device, sealing off the opening of the pocket from outside contamination by saliva and other liquids, and forcing the hydrogel containing preservative, antimicrobial, or wound healing agent into the pocket entrance.

By encapsulating the gingival crevice, which is the entrance to the periodontal pocket, the oral appliance assures that the captured fluids, which are those fluids coating and surrounding the teeth and soft tissues when the tray is inserted, are pushed away from the crevice entrance and kept away by the encapsulating hydrogel over the crevice. Further, as the hydrogel is emptied of antimicrobial, Gingival Crevicular Fluids (GCFs) are wicked up and may be absorbed by the hydrogel in a fluid exchange, removing this contaminated exudate from the infected periodontium. GCFs include exudate fluids containing bacteria, dead cellular structures, interstitial fluids, inflammatory factors, etc. The greater the degree of inflammation due to the periodontal disease, the greater the rate of flow of the GCF. Removing the GCF from the pocket creates space for the preservative, antimicrobial, or wound healing agent and/or hydrogel to occupy. This will result in decreased inflammation and thus decreased GCF flow. As the gingiva at the top of the pocket begins to respond to the medicament (e.g., antimicrobial or preservative), the surface inflammation will decrease, and the pocket will also shrink in depth. Once the treatment regimen by the composition has adequately reduced the pathologic microbiome, other avenues of treatment can be initiated to combat the other aspects of the periodontal disease sequence.

FIG. 5 illustrates an enlarged cross-sectional view of a portion of the oral appliance 400 that can be used in the top down approach. In the embodiment shown, antimicrobial or preservative (e.g., chlorhexidine (CHX)) is disposed in a hydrogel 404 and the hydrogel is disposed at a discrete region 402 of the oral appliance. The hydrogel is shown in an uncompressed state 405 and when worn with slight pressure, the hydrogel will be compressed against, among other things, the gingival crevice or periodontal pocket causing a seal of the entrance of the gingival crevice or periodontal pocket, which prevents other oral fluids (e.g., saliva, exudates, foreign liquids) from entering the crevice or pocket, which allows release of the antimicrobial or preservative in the gingival crevice or periodontal pocket and allows the hydrogel to absorb or wick fluid from the crevice or pocket. In this embodiment, the hydrogel is disposed at a discrete region of the oral appliance and is sized to be greater than the height, width, and length of the entrance of the periodontal pocket or crevice. In this way, when the device is worn, the hydrogel will contact the entrance of the periodontal pocket or crevice and encapsulate and seal it. In some embodiments, when the device is worn there will be a gap formed between the entrance of the periodontal pocket or crevice (top portion) and the bottom of the pocket by the junctional epithelium that will allow, among other things, the medicament (e.g., preservative, an antimicrobial or a wound healing composition) to leach out of the hydrogel and treat the top of the periodontal pocket.

FIG. 6 illustrates an enlarged cross-sectional view of a portion of the oral appliance 400 being worn that is placed adjacent to the teeth and gums using the top down approach to treating periodontal disease. In the embodiment shown, antimicrobial or preservative (e.g., chlorhexidine (CHX)) is disposed in a hydrogel 404 at a discrete region 402 of the oral appliance. The hydrogel is shown in a compressed state 407, where the device is worn and the hydrogel is compressed against, among other things, the gingival crevice or periodontal pocket causing a seal 413 of the entrance of the gingival crevice or periodontal pocket, which prevents oral fluids (e.g., saliva, exudates, foreign liquids, etc.) from entering the crevice or pocket. There is a gap 415 between the junctional epithelium and the entrance 411 of the crevice or pocket, which is now sealed by the hydrogel. This gap allows the hydrogel to release medicament (e.g., a preservative, an antimicrobial or a wound healing agent) in the gingival crevice or periodontal pocket to treat deep down into the inflamed tissue. The medicament (e.g., antimicrobial) release is shown by the down arrows 406. The hydrogel also absorbs or wicks oral fluids from the crevice or pocket which aids healing, shown by the up arrows 408. The hydrogel is placed in the oral appliance and it is configured to create a seal at the entrance of the periodontal pocket so that there will be a bulge of hydrogel at the entrance to cause such a seal 413.

As the medicament (e.g., preservative, an antimicrobial or a wound healing composition) is leached out of the hydrogel, empty hydrogel spaces open up and become available to absorb and remove crevicular/sulcular fluids from the environment. In this way, the hydrogel has dual ability to deliver medicament and wicking action to remove crevicular/sulcular fluids from the environment. This dual action of wicking which then creates a negative crevicular fluid flow, allows the medicament (e.g., antimicrobial) under pressure, shown by pressure points A, B and C, to enter the top portion of the pocket to fill the resultant negative pressure void, thus inserting the medicament (e.g., antimicrobial) further into the pockets. Over sustained daily treatment regimens, the inflammation at the top of the pocket decreases and with decreased inflammation there is decreased swelling and therefore decreased pocket depth.

In some embodiments, the oral appliance has a thickness of from about 0.03 inches to about 0.2 inches. In some embodiments, the oral appliance has a uniform thickness or a non-uniform thickness ranging from about 30.03, 0.04, 0.05, 0.06, 0.07, 0.08, 0.09, 0.10, 0.11, 0.12, 0.13, 0.14, 0.15, 0.16, 0.17, 0.18, 0.19, to about 0.2 inches. The oral appliance can have a uniform or non-uniform thickness of about 0.2 to about 0.5 inches. In some embodiments, the oral appliance comprises a semi-solid construction.

### Oral Wounds

The hydrogel with or without medicament can be used to treat systemic and local conditions. For example, the medicament (e.g., a preservative, an antimicrobial or a wound healing composition) in the hydrogel, such as chlorhexidine, of the current application can be used to treat oral wounds and/or periodontal disease.

The hydrogel having the medicament (e.g., chlorhexidine) forms a protective barrier between the wound and prevents or reduces further irritation and contamination of the oral wound. In some embodiments, the hydrogel forms a protective biofilm that also allows the oral wound to heal.

Oral wounds can be painful to the patient and leave the affected oral tissue raw. This is particularly so when the oral cavity has implants, periodontal disease, laser and graft procedures performed, tooth extraction, dentures and braces, as well as mucosal ulcers, sores, lesions and trauma as well as other injuries to the mouth. Oral wounds include, for example, any lesion, candidiasis (e.g., pseudomembranous candidiasis, chronic atrophic candidiasis, etc.), angular cheilosis, herpectic, gingivostomatits, herpes labialis, aphthous stomatitis (e.g., canker sores), erythema migrans, hairy tongue, lichen planus, periodontal disease, gingivitis, xerostomia, oral carcinoma, oral pyogenic granulomas, oral fibromas, pain and sores resulting from deep tooth scaling, and the like. Oral candidiasis may present as pseudomembranous candidiasis, glossitis, or perlèche (angular cheilitis). Oral candidiasis may signify immune deficiency or other illness. Herpes labialis typically is a mild, self-limited condition. Recurrent aphthous stomatitis most often is a mild condition; however, severe cases may be caused by nutritional deficiencies, autoimmune disorders, or human immunodeficiency virus infection. Erythema migrans is a waxing and waning disorder of unknown etiology. Hairy tongue represents elongation and hypertrophy of the filiform papillae and most often occurs in persons who smoke heavily. Oral lichen planus is a chronic inflammatory condition that may be reticular or erosive. Certain risk factors have been associated with each of these lesions, such as poor oral hygiene, age, tobacco use, and alcohol consumption, and some systemic conditions may have oral manifestations.

Generally, the goal of periodontal treatment is to decrease inflammation and maintain a reduced inflammatory state. Treating periodontitis with traditional oral hygiene regimens (brushing/flossing) is challenging, as the periodontal pockets are difficult to access. Treating the condition therefore requires, in some cases, a scaling and root planning (SRP) procedure, an invasive in-office mechanical debridement of the periodontal pockets. SRP may be effective, but in practical terms receives patient resistance due to the pain, time, and cost involved. There are no longer-term post-SRP periodontal maintenance therapies that practitioners can prescribe to promote further healing and allow for the proper ongoing management of the chronic condition other than good oral hygiene and antimicrobial rinses. This can be a concerning issue as many patients cannot achieve the level of oral hygiene necessary to maintain periodontal health, and long-term use of antimicrobial rinses is limited due to known side effects. Moreover, it is now understood that oral health is no longer just about killing pathogenic bacteria but rather about maintaining an overall healthy balance of the oral microbiota; rinses that indiscriminately kill bacteria therefore may be deleterious. Given the prevalence of periodontal disease, aging populations, and the increased knowledge of the oral-systemic health connection, there is an urgent need for a non-invasive, easy to administer, clinically effective option to treat and manage periodontal disease. The oral appliance of the current application addresses this need.

Sometimes, oral wounds can appear as masses, prompting concern about oral carcinoma. Many are benign, although some (e.g., leukoplakia) may represent neoplasia or cancer. Palatal and mandibular tori are bony protuberances and are benign anomalies. Oral pyogenic granulomas can appear in response to local irritation, trauma, or hormonal changes. Mucoceles represent mucin spillage into the oral soft tissues resulting from rupture of a salivary gland duct. Oral fibromas can form as a result of irritation or masticatory trauma, especially along the buccal occlusal line. Oral cancer may appear clinically as a subtle mucosal change or as an obvious mass. Oral leukoplakia is the most common premalignant oral lesion. Tobacco and heavy alcohol use are the principal risk factors for oral cancer.

Some risk factors that can reduce or prevent oral wound healing include local factors, for example, size of the wound, wound localization, postoperative bleeding, thermal damage, perforation of the sinus maxillary, sharp oral bone tissue edges, local anesthesia, infection (e.g., periodontal disease) hypo-perfusion, ischemia, foreign body, smoking, venous insufficient, mechanical trauma, local toxins, head or neck irradiation, cancer of the mouth, presence of necrotic tissue, edema, or the like. General risk factors that can reduce or prevent oral wound healing include, for example, hereditary defects of wound healing, nutritional deficiency, HIV or other immune disorders, cancer, old age, diabetes, jaundice, alcoholism, uremia, immunosuppressive agents, prolonged use of corticosteroids, chemotherapy, antiresorptive medication, vitamin A, hypothyroidism, anemia, hyperbaric oxygen treatment or the like.

The hydrogel composition containing medicament (e.g., chlorhexidine) of the current application can be used in patient populations that have these risk factors.

### Methods of Making the Composition

In some embodiments, there is a method of making a composition for treating an oral cavity in a patient in need thereof, the method comprising mixing in any order about 12% to about 19% w/w, v/v or w/v of polyvinyl alcohol with about 0.001% to about 20% w/w, v/v or w/v of a medicament, and about 5% to about 9% w/w, v/v or w/v glycerol based on a total w/w, v/v or w/v of the composition to make the composition.

The hydrogel composition configured for the delivery via a device or an oral appliance is prepared in a multi-step process. For example, first, a polyvinyl alcohol (PVA) hydrogel formulation is prepared by dissolving PVA in water and subsequent addition of the preservative or antimicrobial such as chlorhexidine (CHG) and then the humectant glycerol. The CHG-impregnated PVA formulation is then dispensed into the channels of the oral appliance. In some embodiments, the PVA, medicament, optional humectant and water can be mixed in any order.

The required amount to be dispensed in the interior surface of the oral appliance is adjusted according to the channel volume for each patient. Once the hydrogel containing the medicament and optional humectant is formed, the formed hydrogel is dispensed in the channel of the oral appliance. Suitable methods and devices for dispensing the hydrogel in an oral appliance are described at WO2021102265A1, filed Nov. 20, 2020 and assigned to Emanate Biomedical Inc. This entire disclosure is herein incorporated by reference.

The dispensing process, in some embodiments, is followed by three freeze-thaw (FT) cycles of −20° C. to room temperature. This can cause, in some embodiments, crosslinking of the hydrogel. At the end of the final freeze cycle, individual devices are placed in a foil pouch and heat sealed. The pouched products undergo a heat treatment process to improve the adhesion of the hydrogel to the dental tray. Following heat-treatment, pouches are placed in a carton, designed to protect the product during transport and shipping. Preparation and dispensing of the PVA hydrogel, the FT cycle and the pouching of devices are performed in an ISO class 7 cleanroom to protect the device from environmental contamination. In some embodiments, the formed hydrogel can be crosslinked using FT and/or using a crosslinking agent, such as for DMPT.

FIG. 7 illustrates, in one embodiment, the process flow 700 in making a custom fit oral appliance (e.g., oral tray) having a channel specifically constructed for receiving the hydrogel and the steps ending with shipping it to the health care provider (e.g., dentist). In the embodiment illustrated, the process starts with oral trays or an oral appliances custom fit to a patient being manufactured 702. In some embodiments, the oral appliance comprises built-in channels that are adjacent to the treatment area (e.g., soft and/or hard oral tissue) and allow the hydrogel to contact the treatment area when the device is worn. In some embodiments, the hydrogel having a medicament comprises a polyvinyl alcohol (PVA) solution and chlorhexidine gluconate (CHG), which is made and stored in the freezer overnight 704. The PVA solution is equilibrated to room temperature for 24 hours before dispensing 706. The required amount of the PVA to be dispensed in each of the oral appliances is calculated based on the channel volume 708 of the custom fit oral appliance. The PVA solution can be dispensed into the channel of the oral appliance and the amount dispensed is verified 710. The oral appliances that are loaded with hydrogel undergo multiple freeze-thaw cycles 712 that allows, in some embodiment, the hydrogel to crosslink without the need for a crosslinking agent. In some embodiments, the oral appliances undergo three freeze-thaw cycles. Every oral appliance is packaged in a pouch and sealed 714. The oral appliances that are packaged undergo heat treatment before testing against defined specifications 716. In some embodiments, the defined specifications include, but are not limited to, visibility, hardness, custom fit geometry of the oral appliances and other physical and non-physical characteristics. After final testing, the sealed and packaged oral appliances are delivered to a healthcare provider (e.g., dentist) or to the patient directly 718.

FIG. 8 illustrates a process flow chart in accordance with an embodiment of the current application. In various embodiments, the process 800 starts with preparing a composition to be delivered to the oral appliance or oral tray. First, a polyvinyl alcohol (PVA) hydrogel composition is prepared by dissolving PVA in distilled water and subsequent addition of chlorhexidine gluconate (CHG) and glycerol to form the hydrogel solution 802. The hydrogel solution is conditioned at −5° C. for 8 hours and thawed back to room temperature in order to adjust its viscosity 804. The CHG-impregnated PVA composition is then dispensed into the channels of the oral tray 806. The required amount to be dispensed is adjusted according to the channel volume of the oral tray for each patient. The dispensing process is followed by three FT cycles of −20° C. to room temperature 808. This allows, in some embodiment, the hydrogel to crosslink without the need for a crosslinking agent. After the final cycle the trays are heat treated at 65° C. for a period of 3 hours 810. After the heat treatment is complete, the trays are packaged 812 and sealed in labeled foil packaging 814 and lot release testing is performed 816. Oral appliances or oral trays that meet all specifications are packaged in labeled shipper boxes with the instructions for use (IFU) before being shipped 818. Oral appliances or oral trays are then delivered to a healthcare provider such as a dentist or the patient directly.

The present application illustrates different embodiments of the current application. For example, a specific hydrogel composition is made containing medicament or preservative, and humectant in a polymer. The hydrogel composition can be placed inside the oral appliance and the polymer can be crosslinked by physical means (e.g., freezing, thawing, etc.) or by chemical means such as adding a crosslinking agent or viscosity modifying agent.

In some embodiments, the oral appliance containing crosslinked hydrogel can be packaged and subjected to heat treatment at from about 50° C., 51, 52, 53, 54, 55, 56, 57, 58, 59, 60, 61, 62, 63, 64, 65, 66, 67, 68, 69, 70, 71, 72, 73, 74, 75, 76, 77, 78, 79, to about 80° C. (e.g., 55° C. to 75° C. for about 1 to about 3 hours). The heat treatment can enhance the hydrogel's adherence to the desired regions of the oral appliance once the hydrogel composition is loaded in the oral appliance.

Having now generally described the invention, the same may be more readily understood through the following reference to the following examples, which are provided by way of illustration and are not intended to limit the present invention unless specified.

### EXAMPLES

### Example 1

The components to make the composition of Example 1 can be obtained, for example, from the manufacturers/suppliers listed in Table A.

TABLE A

| Hydrogel | CAS Number | Supplier |
|---|---|---|
| Distilled Water | 7732-18-5 | VWR |
| PVA | 9002-89-5 | Sigma Aldrich |
| | | (Saint Louis, MO, |
| | | USA) |
| Glycerol | 56-81-5 | Sigma Aldrich |
| | | (Saint Louis, MO, |
| | | USA) |
| Chlorhexidine | 18472-51-0 | Cyalume Specialty |
| digluconate | | Products (Bound |
| | | Brook, NJ, USA) |

Example 1 is directed to an embodiment of the composition. The composition has chlorhexidine (CHG) as a preservative at 0.05% w/w of the composition. Glycerol is a humectant for preserving the water content of the composition and prolongs the storage duration of the composition at 7.3% w/w of the composition. The hydrogel in the composition is polyvinyl alcohol (PVA) at 15.44% w/w of the composition and distilled water at 77.21% w/w of the composition. The hydrogel also serves as base material forming a matrix and general shape of the composition. Components of the composition in Example 1 are shown in Table 1 below.

TABLE 1
Composition

| Material | Content (w/w) | Range (w/w) | Purpose |
|---|---|---|---|
| Water | 77.21% | 65%-90% | Matrix |
| PVA | 15.44% | 13%-21% | Matrix |
| Glycerol | 7.3% | 2%-9% | Humectant |
| CHG | 0.05% | 0.005%-1% | Preservative |

The composition contains about 0.02-0.07% w/w chlorhexidine The composition, particularly, the hydrogel is capable of swelling, and absorbing additional fluid from about 4% to about 50% w/w. The release specification is shown below in Table 1-1 and the shelf-life assessment is shown in Table 1-2.

TABLE 1-1
Release Specification

| | Specification/Acceptance | |
|---|---|---|
| Description | Criteria | |
| Water Content | >65% | w/w |
| Swelling | 4%-50% | |
| Chlorhexidine Content | 0.02-0.07% | w/w |

TABLE 1-2
Shelf-Life Assessment

| | # of | | | |
|---|---|---|---|---|
| | Devices | | | |
| | (oral | | | |
| | trays) | Sample | | Test |
| Parameter | tested | description | Specification | Result |
| Appearance | 14 | Oral Appliance | Hydrogel must | Pass |
| | | | remain in | |
| | | | place | |
| Hydrogel | 14 | The hydrogel | 4-50% | Pass |
| swelling | | component of the | | |
| | | largest trays¹ | | |
| Hydrogel water | 14 | The hydrogel | >65% w/w | Pass |
| content | | component of the | | |
| | | largest trays | | |
| Chlorhexidine | 14 | The hydrogel | 0.02-0.07% | Pass |
| content w/w % | | component of the | w/w | |
| | | largest trays | | |
| Preservative | 30 | Hydrogel | TBD | |
| efficacy testing | | components were | | |
| | | made in 1″ × 1.5″ | | |
| | | molds and packaged | | |
| | | with larges trays and | | |
| | | packaged in foil | | |
| | | pouch² | | |
| Package | 29 | Worst case | Gross Leak | Pass |
| Integrity | | (largest trays) | Detection with | |
| | | devices packaged | no bubbles | |
| | | in foil pouch | observed | |
| Seal Peel | 29 | Worst case | ≥1.0 lbf | Pass |
| Strength | | (largest trays) | | |
| | | devices packaged | | |
| | | in foil pouch | | |

¹The final finished largest devices were prepared and were subjected to shipping conditioning and accelerated ageing. Prior to testing the pouches were opened and the hydrogel component of the trays were removed from the channels for further testing.
²Due to the narrow and uneven shape of the hydrogels formed in the channels, inoculation of the hydrogel for the purposes of the preservative testing was not possible. Therefore, the amount of hydrogel that would have been deposited within the channel of the largest tray was poured into flat molds and was subjected to the exact sample processing as normal hydrogels. The flat hydrogels (two representing upper and lower jaws) were packaged with two largest trays before further processing.

### Example 2

Example 2 is directed to the performance test of the composition on antimicrobial efficacy with varying chlorhexidine gluconate (CHG) doses, as shown in Table 2 and Table 2-1 below.

The composition is tested with 0.005%, 0.025%, 0.05%, 0.1%, and 0.15% w/w of chlorhexidine (CHG) all of which showed inhibitory effects in the Zone of Inhibition (ZOI) testing. The oral appliance showed higher efficacy towards gram positive bacteria compared to gram negative bacteria, due to the positive charge of CHG. CHG concentrations 0.05% w/w showed efficacy in the modified AATCC 100 test against Staphylococcus aureus and Pseudomonas aeruginosa by reducing the concentration of microorganisms by more than 99.99%. after 30 min contact time. CHG concentrations 0.1% w/w showed efficacy in the modified AATCC 100 test against Staphylococcus aureus and Pseudomonas aeruginosa by reducing the concentration of the microorganisms by more than 99.99%. after 60 min and 24 hour contact time. CHG concentrations 0.005%, 0.05%, and 0.1% w/w were tested against Candida albicans and Candida tropicalis and showed 81.38%, 99.997%, and 99.997% efficacy, respectively. The CHG concentration of 0.05% w/w was the lowest dose showing strong efficacy against both bacteria and fungus and was therefore selected as the suitable preservative concentration.

TABLE 2
Results of zone of inhibition testing of with
various CHG doses over a 24-hour period.

| | | Average width of |
|---|---|---|
| | Oral Appliance | Clear Zone of |
| Microorganism | dose (% w/w) | Inhibition (mm) |
| S. aureus ATCC 6538 | 0.05 | 5 |
| | 0.1 | 4.5 |
| | 0.15 | 5 |
| E. coli ATCC 6538 | 0.05 | 2.5 |
| | 0.1 | 3 |
| | 0.15 | 3 |
| S. aureus ATCC 6538 | 0.005 | 7.5 |
| | 0.025 | 8 |
| | 0.05 | 8.5 |
| E. coli ATCC 6538 | 0.005 | 2 |
| | 0.025 | 3.5 |
| | 0.005 | 6.5 |

TABLE 2-1
Result of antimicrobial efficacy testing for various doses of CHG

| | | | | | Percent | Log₁₀ |
|---|---|---|---|---|---|---|
| | | CHG | Contact | | Reduction | Reduction |
| Study | Test | Conc | time | Average | Compared to | Compared |
| number | microorganism | (% w/w) | (min) | CFU/carrier | Control | to Control |
| NG17964 | C. albicans | 0.005 | 30 | 1.00E+05 | 43.52% | 0.25 |
| | ATCC 10231 | 0.05 | | 2.48E+02 | 99.86% | 2.86 |
| | | 0.1 | | 7.50E+00 | 99.996% | 4.37 |
| | C. tropicalis | 0.005 | 30 | 2.70E+04 | 81.38% | 0.73 |
| | ATCC 750 | 0.05 | | <5.00E+00¹ | >99.997% | >4.46 |
| | | 0.1 | | <5.00E+00¹ | >99.997% | >4.46 |
| NG17598-A1 | S. aureus ATCC | 0.005 | 30 | <5.00E+00¹ | >99.996% | >4.40 |
| | 6538 | | | | | |
| | P. aeruginosa | | 30 | <5.00E+00¹ | >99.996% | >4.43 |
| | ATCC 9027 | | | | | |
| | S. aureus ATCC | 0.1 | 60 | <5.00E+00¹ | >99.997% | >4.57 |
| | 6538 | | 24 (hrs) | <5.00E+00¹ | >99.997% | >4.58 |
| | P. aeruginosa | | 60 | <5.00E+00¹ | >99.996% | >4.53 |
| | ATCC 9027 | | 24 (hrs) | <5.00E+00¹ | >99.998% | >4.70 |

¹Limit of detection. Values fell below the limit of detection <5.00E+00.

### Example 3

Example 3 is directed to the manufacture of the oral appliance containing the composition. In summary, polyvinyl alcohol (PVA) is first dissolved in distilled water followed by the addition of CHG and glycerol (hydrogel solution). The hydrogel solution is conditioned at −5° C. for 8 hours and thawed back to room temperature in order to adjust its viscosity. The hydrogel solution is then dispensed within the preformed channels of the dental tray. The amount of the hydrogel dispensed is determined by the surface area of the channel of the oral appliance, measured for every patient. The oral appliances are then subjected to three freeze-thaw (FT) cycles of −20° C. to room temperature. After the final cycle, the trays are heat treated at 65° C. for a period of 3 hours. After the heat treatment is complete, the oral appliances are packaged and sealed in labeled foil packaging and release testing is performed. Devices that meet all specifications are packaged in labeled shipper boxes with the instructions for use (IFU) before being shipped. The process flow to these embodiments are shown in FIG. 7 and FIG. 8.

### Example 4

Example 4 is directed to the viscosity test conducted on the composition before the polymer of the hydrogel is crosslinked. Multiple samples of the compositions were tested using a CPE-51 spindle at a temperature of 25 degrees Celsius. The viscosity results are summarized in Table 3 below, which summarize the results of Table 3-1 also below.

TABLE 3
Summary Table of the Viscosity Test Results of Table 3-1

| | | TEMPERATURE | AVG VISCOSITY |
|---|---|---|---|
| LABEL | SPINDLE | (° C.) | (cP₁) |
| S1 Specimen 1 | CPE-51 | 25 | 5031.0 |
| S1 Specimen 2 | CPE-51 | 25 | 5041.47 |
| S1 Specimen 3 | CPE-51 | 25 | 5285.1 |

Specifically, the test standard ASTM D1084 was followed, deviating only in the use of the cone and plate apparatus instead of the rotational viscometer. Viscosity measurements were taken in triplicate. Measurements for the sample were performed using a Brookfield RVDVII Cone and Plate viscometer fitted with a CPE-51 spindle. A 0.5 ml aliquot of sample was utilized for analysis and was equilibrated to 25° C. via a water bath prior to measurement. Measurements were taken at 3 different sheer rates: one producing a “low” percent torque (% T) (e.g., 10-30% T), one producing a “medium” % T (e.g., 30-70% T), and one producing a “high” % T (e.g., 70-90% T), as shown in Table 3-1 below.

TABLE 3-1
Summary of Brookfield Viscosity Results

| | | | | | | | | AVG |
|---|---|---|---|---|---|---|---|---|
| | | TEMPERATURE | | | | FSM1 | VISCOSITY | VISCOSITY |
| LABEL | SPINDLE | (° C.) | REPLICATE | RPM | % T | (CP2) | (CP) | (CP) |
| S1 | CPE- | 25 | 1 | 2 | 18.8% | 28,890 | 5431.3 | 5031.0 |
| Specimen | 51 | | 2 | 5 | 48.3% | 10,356 | 5001.9 | |
| 1 | | | 2 | 5 | 48.3% | 10,356 | 5001.9 | |
| S1 | CPE- | 25 | 1 | 2 | 19.1% | 28,890 | 5518.0 | 5041.8 |
| Specimen | 51 | | 2 | 5 | 47.4% | 10,356 | 4908.7 | |
| 2 | | | 3 | 8 | 72.6% | 6,472 | 4698.7 | |
| S1 | CPE- | 25 | 1 | 2 | 20.3% | 28,890 | 5864.7 | 5285.1 |
| Specimen | 51 | | 2 | 5 | 50.1% | 10,356 | 5188.4 | |
| 3 | | | 3 | 8 | 74.2% | 6,472 | 4802.2 | |

While particular embodiments of the present disclosure have been shown and described, it will be appreciated by those skilled in the art that, based upon the teachings herein, changes and modifications may be made without departing from this disclosure and its broader aspects and, therefore, the appended claims are to encompass within their scope all such changes and modifications as are within the true spirit and scope of this disclosure. The true spirit and scope is considered to encompass devices and processes, unless specifically limited to distinguish from known subject matter, which provide equivalent functions as required for interaction with other elements of the claims and the scope is not considered limited to devices and functions currently in existence where future developments may supplant usage of currently available devices and processes yet provide the functioning required for interaction with other claim elements.

1. A composition for treating the oral cavity in a patient in need thereof, the composition comprising a medicament, a polyvinyl alcohol (PVA), and optionally a humectant, the medicament in an amount of about 0.001% w/w, v/v or w/v to about 5% w/w, v/v or w/v, the polyvinyl alcohol in an amount of about 5% w/w, v/v or w/v to about 99% w/w, v/v or w/v, the optional humectant in an amount of about 0.5% w/w, v/v or w/v to about 15% w/w, v/v or w/v based on a total w/w, v/v or w/v of the composition.

2. The composition of claim 1, wherein the medicament is chlorhexidine.

3. (canceled)

4. The composition of claim 1, wherein the medicament is chlorhexidine gluconate in an amount (i) from about 1% to about 5% w/w, v/v or w/v based on the total w/w, v/v or w/v of the composition; (ii) from about 0.0001% to about 1% w/w, v/v or w/v based on the total w/w, v/v or w/v of the composition; or (iii) from about 0.025% to about 1% w/w based on the total w/w of the composition.

5. The composition of claim 1, wherein the polyvinyl alcohol (PVA) is in an amount from about 12% to about 19% w/w, v/v or w/v based on the total w/w, v/v or w/v of the composition.

6. The composition of claim 1, wherein the composition comprises water or water in the polyvinyl alcohol (PVA) in an amount from about 61% to about 90% w/w, v/v or w/v based on the total w/w, v/v or w/v of the composition.

7. The composition of claim 6, wherein the humectant is glycerol.

8. The composition of claim 7, wherein the medicament comprises chlorhexidine gluconate in an amount of about 0.025% w/w to about 0.1% w/w, the polyvinyl alcohol (PVA) in an amount of about 10% w/w to about 20% w/w, the glycerol in an amount of about 5% w/w to about 10% w/w and water in the composition or water in the polyvinyl alcohol (PVA) in an amount of 50% w/w to about 90% w/w based on the total weight of the composition.

9. The composition of claim 1, wherein the medicament is chlorhexidine gluconate configured to contact teeth and/or gingival soft tissues of the oral cavity for about 5 minutes to about 40 minutes.

10. The composition of claim 1, wherein the medicament is chlorhexidine gluconate configured to contact teeth and/or gingival soft tissues of the oral cavity for about 30 minutes to about 24 hours.

11. The composition of claim 1, wherein the medicament is chlorhexidine gluconate in an amount of about 0.05% to about 0.06% w/w, v/v or w/v based on the total w/w, v/v or w/v of the composition.

12. The composition of claim 1, wherein the humectant is glycerol in an amount of about 7.3% w/w, v/v or w/v based on the total w/w, v/v or w/v of the composition.

13. (canceled)

14. The composition of claim 1, wherein the polyvinyl alcohol (PVA) is in an amount of about 15% to about 16% w/w, v/v or w/v, and water is in the composition or is in the polyvinyl alcohol (PVA) in an amount of about 77% w/w, v/v or w/v based on the total w/w, v/v or w/v of the composition.

15. The composition of claim 2, wherein the chlorhexidine is a preservative agent.

16. The composition of claim 2, wherein the chlorhexidine is an antimicrobial agent.

17. (canceled)

18. A composition for treating an oral cavity in a patient in need thereof, the composition comprising a medicament, glycerol, polyvinyl alcohol and water in the composition or water in the polyvinyl alcohol, the medicament being in an amount of about 0.001% to about 20% w/w, v/v or w/v, the glycerol being in an amount of from about 5% to about 9% w/w, v/v or w/v, polyvinyl alcohol being in an amount from about 12% to about 19% w/w, v/v or w/v, and water being in the composition or in the polyvinyl alcohol in an amount from about 61% to about 82% w/w, v/v or w/v based on a total w/w, v/v or w/v of the composition.

19. The composition of claim 18, wherein (i) the composition is configured to be disposed in an oral appliance, the oral appliance configured to hold the composition in contact with tissue in the oral cavity; and/or (ii) the oral appliance is configured to hold the composition so as to encapsulate the gingival crevice.

20. (canceled)

21. (canceled)

22. A method of making a composition for treating an oral cavity in a patient in need thereof, the method comprising mixing in any order about 12% to about 19% w/w, v/v or w/v of polyvinyl alcohol with about 0.001% to about 20% w/w, v/v or w/v of a medicament, and about 5% to about 9% w/w, v/v or w/v glycerol based on a total w/w, v/v or w/v of the composition to make the composition.

23. The method of claim 22, wherein the polyvinyl alcohol is crosslinked using a chemical agent.

24. The method of claim 22, wherein after mixing, the polyvinyl alcohol is crosslinked by freezing and then thawing the polyvinyl alcohol.

25. The method of claim 22, wherein after mixing, the polyvinyl alcohol is crosslinked to maintain its shape and location within the oral appliance.

26.-58. (canceled)
